(19) United States
(12) Patent Application Publication
(10) Pub. No.: US 2024/0096455 A1
(43) Pub. Date: Mar. 21, 2024

(54) SYSTEMS AND METHODS FOR AUTOMATIC HEALTH DATA PROCESSING

(71) Applicant: Optum, Inc., Minnetonka, MN (US)

(72) Inventors: Christian M. HOLES, Deerfield Beach, FL (US); Pavan K. KURAM, Plymouth, MN (US); Lawrence D. GARBER, Southborough, MA (US); Jennifer M. PIKE, Northfield, MN (US)

(73) Assignee: Optum, Inc., Minnetonka, MN (US)

(21) Appl. No.: 18/175,057

(22) Filed: Feb. 27, 2023

Related U.S. Application Data
(60) Provisional application No. 63/376,136, filed on Sep. 19, 2022.

(51) Int. Cl. G16H 10/20; G06F 40/30; G16H 10/60

(52) U.S. Cl. CPC G16H 10/20; G06F 40/30; G16H 10/60

(57) ABSTRACT

Systems and methods for automatic health data processing are disclosed. One or more complaints are extracted from question and answer data of an automated patient interview. A parent-child symptom mapping is referenced to identify a respective parent symptom corresponding to each complaint. Based on the question and answer data, a presence or absence of evidence of the child symptoms associated with each identified parent symptom is determined. The child symptoms are categorized into a positive or negative category based on the determination. An object is generated that includes, for each of the complaints, a positive and negative list of any of the child symptoms included in the positive and negative category, respectively. The object is processed to generate a history of present illness (HPI) portion of a notation in a graphical format. The notation is stored in the patient's electronic health record, and a healthcare provider notification is transmitted.

### CROSS-REFERENCE TO RELATED APPLICATION(S)

This application claims the benefit of priority from U.S. Provisional Application No. 63/376,136, filed on Sep. 19, 2022, which is incorporated by reference herein in its entirety.

### TECHNICAL FIELD

The present disclosure relates generally to systems and methods for health data processing, and more particularly, to systems and methods for processing question and answer data from an automated patient interview.

### BACKGROUND

Physician burnout is significantly higher than other job categories, and experts link this fact to clinicians spending more than half of their time entering documentation and performing tasks in the electronic health record system. They spend only a quarter of their time seeing patients, which is what they would prefer to be doing. Patients are also frustrated with having to repeat their symptoms over and over to different members of the healthcare team, and wanting more time to speak with the physician during their visits.

Advancements in technology have facilitated increased automation in the collection and storage of patient health data. For example, several conventional healthcare applications have incorporated symptom reporting features to automatically capture current patient symptoms that are saved as patient health data to the patient's electronic health record for review by the healthcare team. However, the format in which the patient health data is captured and stored by these conventional applications is not easily consumable by healthcare providers, and thus fails to save healthcare providers time interacting with the patient's electronic health record and/or mitigate the underlying burnout. For example, the current patient symptoms are typically captured by the conventional healthcare applications in a question and answer format that is stored as a transcript in the electronic health record. Such a format however does not enable conventional healthcare applications to efficiently process the underlying information to provide insights that are easily consumable by healthcare providers, thus requiring the healthcare team members to read an entirety of the transcript to distill the patient's current history of present illness (HPI).

The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY

The techniques of this disclosure improve the state of conventional healthcare applications. In one aspect, computer-implemented methods for automatic health data processing are disclosed herein. An example method includes receiving, by one or more processors, question and answer data from an automated interview of a patient, extracting, by the one or more processors, one or more complaints from the question and answer data, and referencing, by the one or more processors, a mapping that includes a plurality of parent symptoms and a plurality of child symptoms associated with the plurality of parent symptoms to identify a respective parent symptom from the plurality of parent symptoms corresponding to each of the one or more complaints. The method also includes determining, by the one or more processors and based on the question and answer data, a presence or absence of evidence of one or more child symptoms, among the plurality of child symptoms, associated with each identified parent symptom, categorizing, by the one or more processors, each of the one or more child symptoms into a positive category or a negative category based on the determination, and generating, by the one or more processors, an object that includes, for each of the one or more complaints, (a) a positive list including any of the one or more child symptoms included in the positive category and (b) a negative list including any of the one or more child symptoms included in the negative category. The method further includes processing, by the one or more processors, the object to generate a history of present illness (HPI) portion of a notation for the patient in a graphical format, storing the notation in an electronic health record of the patient, and generating and transmitting, to a healthcare provider application, a notification that includes one or more indications of an occurrence of the automated interview and availability of the notation in the electronic health record for access.

In accordance with another aspect, systems for automatic health data processing are disclosed herein. An example system includes one or more processors, and at least one memory storing instructions that, when executed by the one or more processors, cause the one or more processors to perform operations. The operations include receiving question and answer data from an automated interview of a patient, extracting one or more complaints from the question and answer data, and referencing a mapping that includes a plurality of parent symptoms and a plurality of child symptoms associated with each of the plurality of parent symptoms to identify a respective parent symptom from the plurality of parent symptoms corresponding to each of the one or more complaints. The operations also include determining, based on the question and answer data, a presence or absence of evidence of one or more child symptoms, among the plurality of child symptoms, associated with each identified parent symptom, categorizing each of the one or more child symptoms into a positive category or a negative category based on the determination, and generating an object that includes, for each of the one or more complaints, (a) a positive list including any of the one or more child symptoms included in the positive category and (b) a negative list including any of the one or more child symptoms included in the negative category. The operations further include processing the object to generate a HPI portion of a notation for the patient in a graphical format, storing the notation in an electronic health record of the patient, and generating and transmitting, to a healthcare provider application, a notification that includes one or more indications of an occurrence of the automated interview and availability of the notation in the electronic health record for access.

In accordance with a further aspect, non-transitory computer readable media for automatic health data processing are disclosed herein. An example non-transitory computer readable medium stores instructions which, when executed by one or more processors, cause the one or more processors to perform operations. The operations include receiving question and answer data from an automated interview of a patient, extracting one or more complaints from the question and answer data, and referencing a mapping that includes a plurality of parent symptoms and a plurality of child symptoms associated with each of the plurality of parent symptoms to identify a respective parent symptom from the plurality of parent symptoms corresponding to each of the one or more complaints. The operations also include determining, based on the question and answer data, a presence or absence of evidence of one or more child symptoms, among the plurality of child symptoms, associated with each identified parent symptom, categorizing each of the one or more child symptoms into a positive category or a negative category based on the determination, and generating an object that includes, for each of the one or more complaints, (a) a positive list including any of the one or more child symptoms included in the positive category and (b) a negative list including any of the one or more child symptoms included in the negative category. The operations further include processing the object to generate a history of present illness (HPI) portion of a notation for the patient in a graphical format, storing the notation in an electronic health record of the patient, and generating and transmitting, to a healthcare provider application, a notification that includes one or more indications of an occurrence of the automated interview and availability of the notation in the electronic health record for access.

### BRIEF DESCRIPTION OF THE DRAWINGS

The accompanying drawings, which are incorporated in and constitute a part of this specification, illustrate various example embodiments and together with the description, serve to explain the principles of the disclosed embodiments.

FIG. 1 is a diagram showing an example of an environment for automatic health data processing to generate a notation, according to some embodiments of the disclosure.

FIG. 2 is a system flow diagram showing an example of a process for automatic health data processing, according to some embodiments of the disclosure.

FIG. 3 is a block diagram showing an example of a process for training and using a machine learning model to output a healthcare-related recommendation based on patient interview data, according to some embodiments of the disclosure.

FIG. 4 is a flowchart showing an example of a process for generating a notation, according to some embodiments of the disclosure.

FIG. 5 is a flowchart showing an example of a process for building a history of present illness (HPI) object, according to some embodiments of the disclosure.

FIGS. 6A-60 show examples of multiple patient interview user interfaces, according to some embodiments of the disclosure.

FIGS. 7A-7F show examples of multiple scheduling user interfaces, according to some embodiments of the disclosure.

FIG. 8 shows an example of an HPI portion of a notation, according to some embodiments of the disclosure.

FIG. 9 illustrates an implementation of a computer system that executes techniques presented herein, according to some embodiments of the disclosure.

### DETAILED DESCRIPTION

As briefly mentioned above, patient health data captured and stored by conventional healthcare applications is not easily consumable by healthcare providers, and thus fails to save healthcare providers time interacting with the patient's electronic health record and/or mitigate burnout. For example, patient symptoms captured using symptom reporting features of conventional healthcare applications are typically captured in a question and answer format that is stored as a transcript in the electronic health record. Thus, the healthcare providers are required to read an entirety of the transcript to distill the patient's current history of present illness (HPI).

The present disclosure solves this problem and/or other problems described above or elsewhere in the present disclosure, namely by utilizing a computer technique for converting question and answer data obtained from an automated patient interview into a readable format for storage in an electronic health record. Specifically, question and answer data from an automated interview of a patient is received, and portions of the question and answer data associated with the patient's HPI are identified and converted into a graphical format, such as a chart, table, or other format, for storage as a HPI portion of a notation in the electronic health record.

Compared to conventional healthcare applications that generate and provide a transcript of question and answer data that must be read in its entirety and distilled by the healthcare provider to manually obtain the patient's HPI, the generation of the graphical format of the HPI portion of the notation disclosed herein automatically distills the patient's HPI from the question and answer data using a mapping computer technique further described herein, and presents the patient's HPI (e.g., on a display) in an easily readable format. For example, for each complaint indicated by the patient during the automated patient interview, a series of questions associated with the complaint are presented and answered by the patient, forming a portion of the question and answer data. In some embodiments, the complaint is a parent symptom, and the series of questions are directed to invoking answers as to which child symptoms associated with the parent symptom are being experienced by the patient. The graphical format of the HPI portion of the notation generated using a parent-child symptom mapping technique (hereinafter referred to as simply “parent-child symptom mapping”) displays each complaint identified from the question and answer data, and for each complaint, a positive list and a negative list of child symptoms associated with the parent symptom corresponding to the complaint. The positive list indicates child symptoms being experienced by the patient and the negative list indicates child symptoms not being experienced by the patient based on the question and answer data. Therefore, a healthcare provider accessing the notation within the electronic health record can quickly discern which symptoms are being experienced by a patient (and which are not) by viewing the positive and negative lists included in the HPI portion to facilitate diagnosis, treatment, etc. Resultantly, an amount of healthcare provider interaction time required with the electronic health record is reduced, leaving more interaction time with the patients and mitigating physician burnout, among other advantages described elsewhere herein.

In some embodiments, multi-threading can be performed to process questions from the question and answer data in parallel to increase processing speed and performance of one or more computing devices of the system configured to generate the HPI portion of the notation. Furthermore, computing resources including storage and processing resources can be saved by allowing simplified HPI notations to be stored and retrieved for user viewing, as compared to raw transcripts that may require additional storage space and processing power of one or more computing devices of the system.

In some scenarios and/or embodiments, a patient computing device (e.g., a client device) launches a symptom checker feature of a patient application executing on the patient computing device to initiate the automated patient interview. The patient application is associated with a healthcare services system (e.g., a server device) that the patient interacts with using the patient application to perform healthcare-related activities, such as to view their electronic health record and/or schedule a healthcare visit, in addition to engaging in automated patient interviews. As part of the automated patient interview, an AI-based application bot, for example, presents a series of questions about the patient's symptoms, risk factors, and/or medical history via the patient application to prompt answers from the patient. Possible causes, a recommended level of care, other available levels of care, etc. are provided as example results of the patient interview via the patient application. Along with possible causes, some common treatments (e.g., self-care advice and/or advice when to see a healthcare provider) for each possible cause can be shared via a link or embedded as content associated with the respective possible cause. As part of the patient interview, the patient application prompts the patient to select a level of care as desired. For at least certain levels of care selected (e.g., virtual visits, in person visits, etc.), a healthcare visit is automatically scheduled directly via the patient application.

The question and answer data is recorded by the application bot and shared with a healthcare services system to enable subsequent viewing by the patient's healthcare provider team (e.g., nurses, medical assistants, and doctors) to prevent the patient from having to repeat his or her symptoms, risk factors, etc. when interacting with a healthcare provider. For example, the question and answer data is organized into a medical SOAP (Subjective, Objective, Assessment, Plan) notation that is transferred directly for storage in the patient's electronic health record.

For at least a portion of the question and answer data related to the patient's symptoms (e.g., the portion related to the patient's HPI), a mapping of parent-to-child symptoms is referenced in order to build an object to represent the HPI. For example, a tool is used to iterate through each complaint (e.g., condition) extracted from the question and answer data, and determine whether there is a corresponding parent symptom in the mapping. If there is a corresponding parent symptom, a determination of whether the child symptoms associated with the parent symptom are present or absent in the question and answer data is made, and the child symptoms are categorized into a positive or negative category and included in a positive or negative list, respectively. Positive is a notation to indicate that a child symptom of a parent symptom exists as per the patient response in the question and answer data (e.g., the patient is experiencing the child symptom). Negative is a notation to indicate that the child symptom of the parent symptom does not exist as per the patient response in the question and answer data (e.g., the patient is not experiencing the child symptom). The object is further processed into an easily readable, graphical format for inclusion in the notation, along with other question and answer data that is organized into the medical SOAP. The conversion of the portion of the question and answer data related to the patient's HPI into the graphical format reduces an amount of time it takes for the healthcare provider to read and comprehend the notation (e.g., by about 3 minutes).

The healthcare services system provides a notification to one or more healthcare provider computing devices associated with the healthcare provider team (e.g., via a healthcare provider application) to indicate that the patient has reported symptoms to prompt retrieval of the notation, and cause the healthcare provider team to initiate a process of calling the patient, scheduling an appointment (if not already scheduled by the patient via the patient application), verifying the information, diagnosing, and/or treating the patient. The healthcare provider team uses the question and answer data as a base to, for example, ask new questions to deliver appropriate care, as well as edit or remove any of the question and answer data included in the notation. In some examples, the healthcare provider determines that they can diagnose and treat the patient's condition without talking live, real-time with the patient. Additionally or alternatively, the healthcare provider may utilize the healthcare provider application to send communications to the patient with additional questions or with a recommendation to escalate their level of care (e.g., from the virtual visit recommended by the symptom checker feature to in-person care).

The techniques performed by the systems described herein for generating the HPI portion of the notation in the graphical format address both healthcare provider and patient experiences. For example, the graphical format of the HPI portion of the notation generated by the systems reduces an amount of time healthcare providers spend on reviewing records and/or documenting patient notes (e.g., as compared to time spent when reviewing a conventional question and answer transcript to distill the HPI), which provides healthcare providers more time to focus on the patient and their treatment or be more accessible for other patients. Additionally, the HPI portion of the notation generated by the systems allows healthcare providers to more strategically stratify patients to better align limited resources of the team to the various levels of acuity, such as sending low acuity patients to nurse practitioners instead of the physicians who may be reserved for high acuity patients. Further, the HPI portion of the notation generated by the systems help enable healthcare providers to more quickly decide if they have enough information to diagnose and treat a patient or if they need to ask for more, which ultimately enables healthcare providers to have control over diagnosing, treating, and documenting the patient's care.

Additionally, the techniques performed by the systems described herein for generating the HPI portion of the notation in the graphical format facilitate a more seamless healthcare experience that shares data from the patient interview with the healthcare provider. For example, the generation and inclusion of the HPI portion of the notation in the electronic health record can eliminate the 3-4 times patients on average repeat their symptoms during a healthcare visit as they meet with the various care team members. Additionally, the results of the patient interview provide faster triage (e.g., level of care) recommendations to the patient than contacting a healthcare provider and/or insurance company, as well as remove any onus on the patient to know how their current symptoms relate to particular levels of care. Further, by providing the possible causes of the patient's symptoms and information about how the patient can access care related to those symptoms, healthcare becomes more easily accessible to the patient and allow the patient to have ultimate choice in the level of care they choose.

Further, the techniques performed by the systems described herein educate patients and inform providers in the patients' decision-making process. For example, several options are normally given to both the patient and provider, including an option for patients to meet with the provider to receive care.

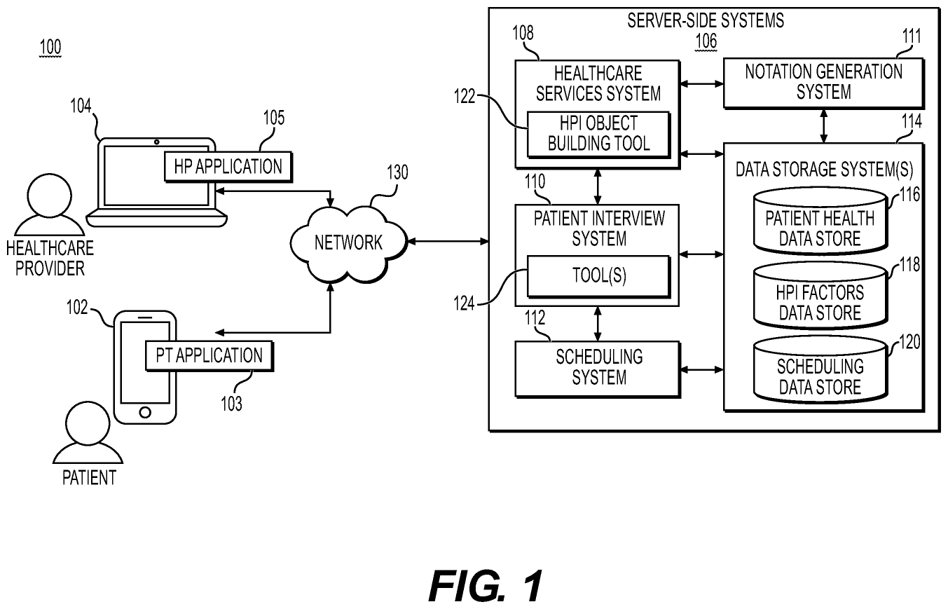

FIG. 1 is a diagram showing an example of an environment 100 for automatic health data processing to generate a notation, according to aspects of the disclosure. A patient computing device 102 and a healthcare provider computing device 104 communicates with one or more of the other components of the environment 100 across a network 130, including one or more server-side systems 106.

The server-side systems 106 include a healthcare services system 108, a patient interview system 110, a notation generation system 111, a scheduling system 112, and/or one or more data storage system(s) 114, among other systems. In some examples, the healthcare services system 108, the patient interview system 110, the notation generation system 111, the scheduling system 112, and/or the data storage system(s) 114 are associated with a common entity, e.g., a common healthcare services provider, such as a care delivery organization (CDO), or the like. In such examples, the healthcare services system 108, the patient interview system 110, the notation generation system 111, the scheduling system 112, and/or the data storage system(s) 114 can be part of a cloud service computer system (e.g., in a data center). That is, the various systems can be components or subsystems of a larger computer system. In other examples, one or more of the patient interview system 110, the notation generation system 111, the scheduling system 112, and/or the data storage system(s) 114 are separate systems associated with different entities. In such examples, each of the separate systems are communicatively connected to one another over the network 130 (e.g., via an application programming interface (API)).

The systems and devices of the environment 100 can communicate in any arrangement. As will be discussed herein, systems and/or devices of the environment 100 communicate in order to: perform and record question and answer data of an automated patient interview, determine patient output data (e.g., possible causes, a recommended level of care, and/or a recommended care plan) based on the question and answer data to provide as results of the patient interview, automatically generate and store a notation in the patient's electronic health record based on the question and answer data, and/or automatically schedule a healthcare visit, among other activities.

The patient computing device 102 is configured to enable a patient to access and/or interact with other systems in the environment 100. For example, the patient computing device 102 is a computer system such as, for example, a desktop computer, a laptop computer, a tablet, a smart cellular phone, a smart watch, or other wearable computer, etc. The patient computing device 102 includes one or more applications, e.g., a program, plugin, browser extension, etc., installed on a memory of the respective computing device. In some embodiments, the applications may be associated with one or more of the other components in the environment 100. For example, the applications include one or more of system control software, system monitoring software, software development tools, etc.

As shown in FIG. 1, at least a portion of one or more instructions stored in a memory of the patient computing device 102 are associated with a patient application 103 (abbreviated as PT application) that is configured to communicate with one or more of the server-side systems 106. For example, the patient application 103 is executed on the patient computing device 102 to communicate with the healthcare services system 108 to launch features of the patient interview system 110 to report symptoms via an automated patient interview and/or features of the scheduling system 112 to schedule a healthcare visit, among other features. The patient application 103 specifically includes features directed toward patients (e.g., as opposed to features directed toward healthcare providers). In some examples, the patient application 103 is a thick client application that is installed locally on the patient computing device 102 (e.g., a desktop application or mobile application). In other examples, the patient application 103 is a thin client application (e.g., a web application) that is rendered via a web browser launched on the patient computing device 102.

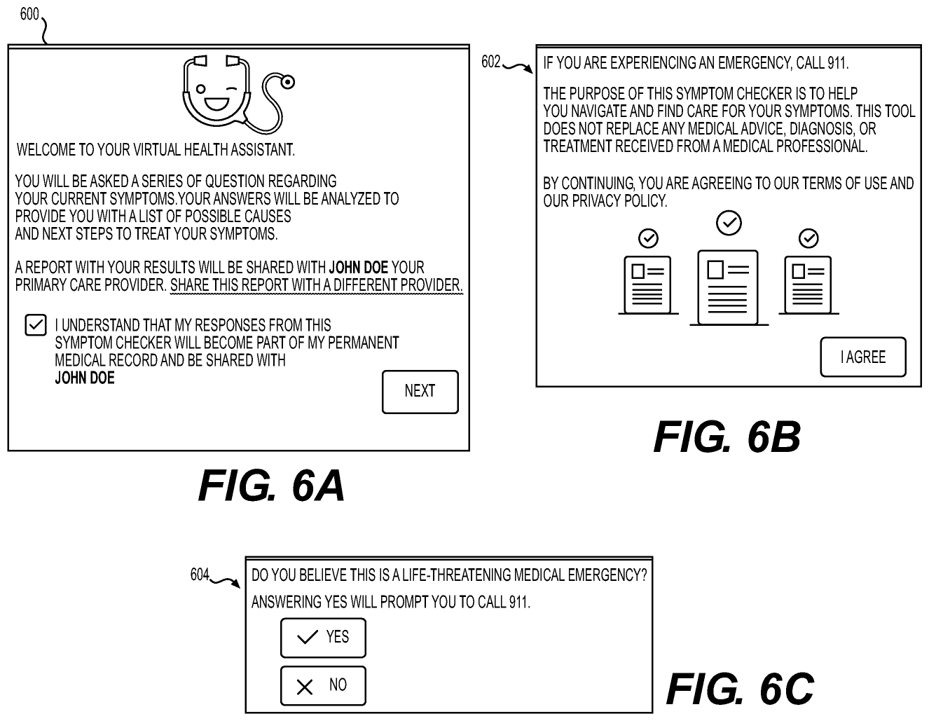

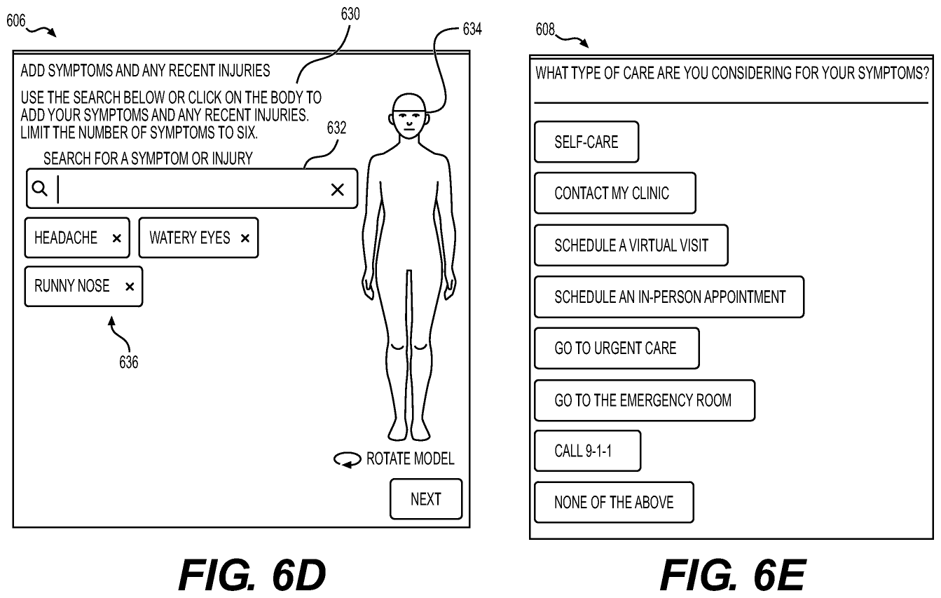

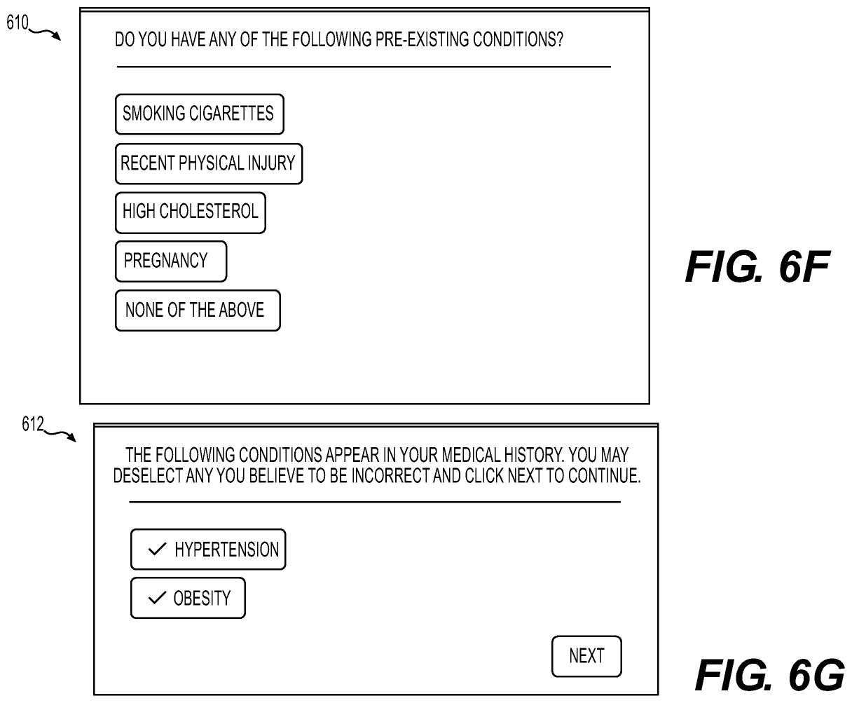

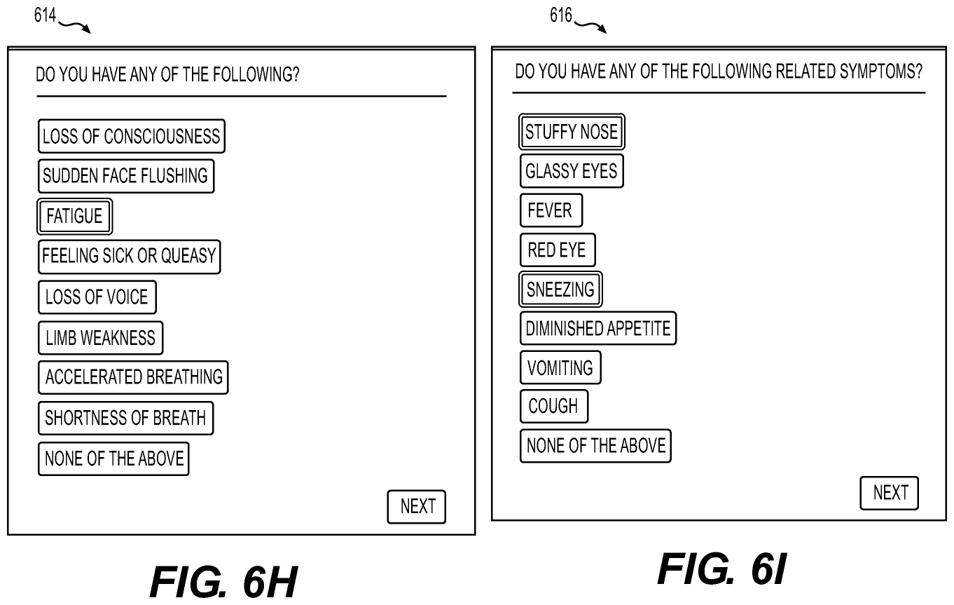

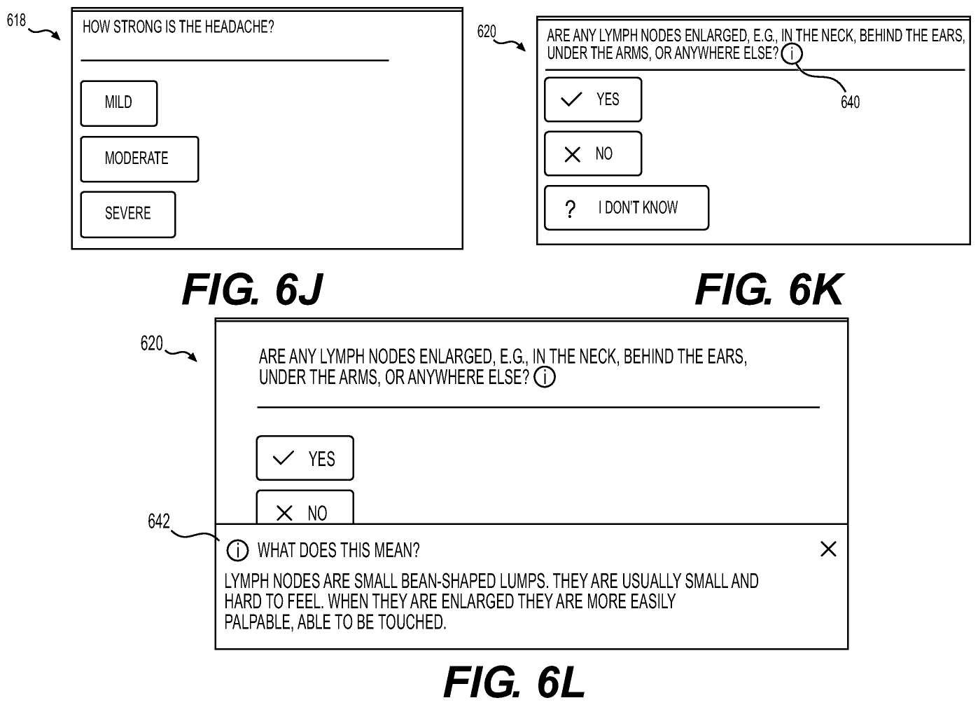

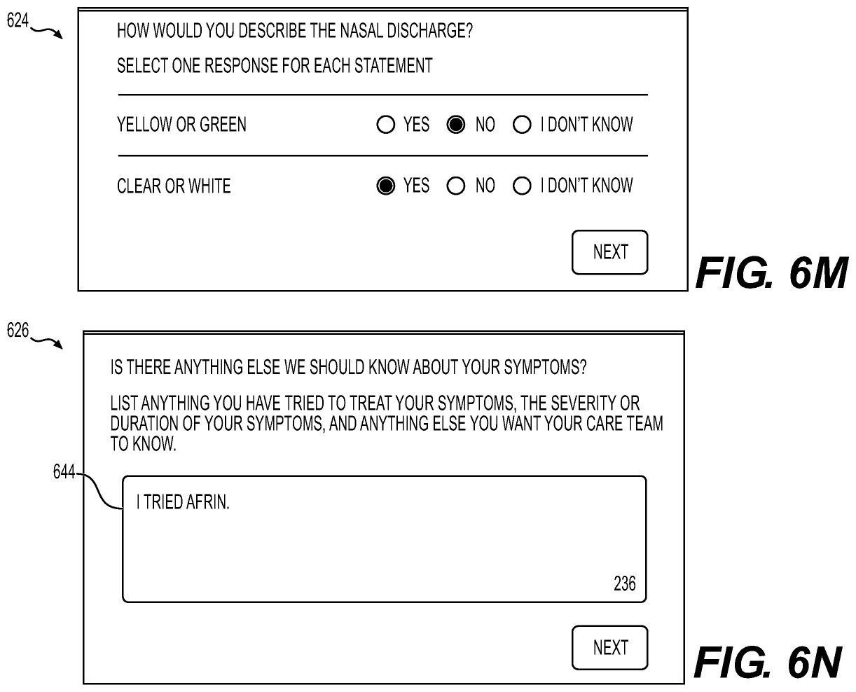

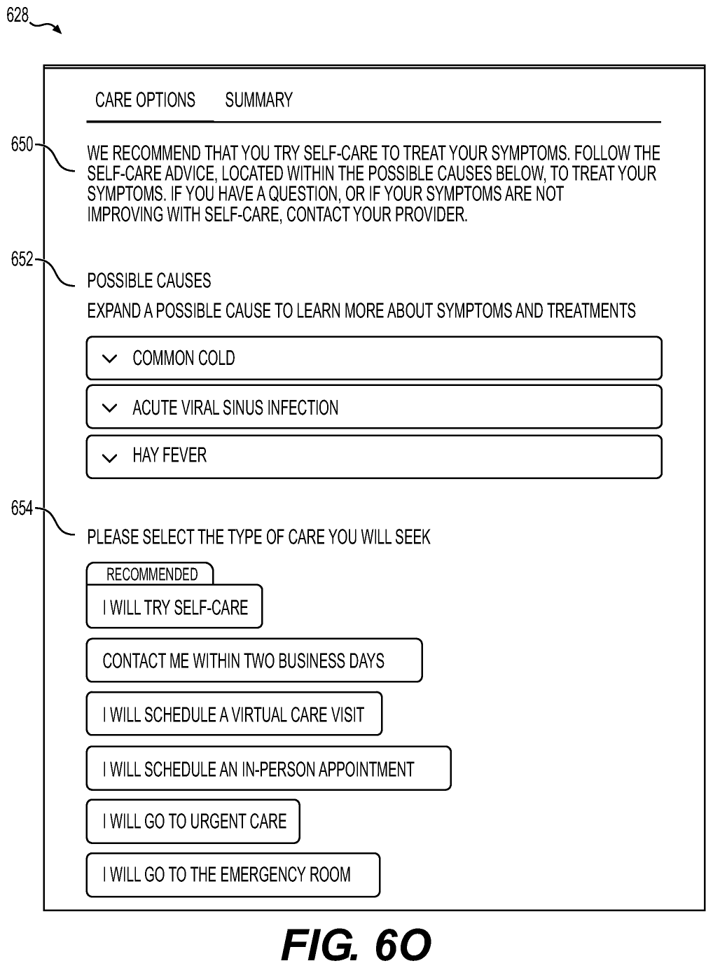

Additionally, one or more components of the patient computing device 102, such as the patient application 103, generate, or cause to be generated, one or more graphic user interfaces (GUIs) based on instructions/information stored in the memory, instructions/information received from the other systems in the environment 100, and/or the like and cause the GUIs to be displayed via a display of the patient computing device 102. The GUIs can be, e.g., mobile application interfaces or browser user interfaces and include text, input text boxes, selection controls, and/or the like. In some examples, the display includes a touch screen or a display with other input systems (e.g., a mouse, keyboard, etc.) for the patient to control the functions of the patient computing device 102. Example GUIs displayed via the patient application 103 executing on the patient computing device 102 are shown in FIGS. 6A-60 and FIGS. 7A-7F.

The healthcare provider computing device 104 is configured to enable a healthcare provider (e.g., a physician, nurse, administrative assistant, etc. of a healthcare team) to access and/or interact with other systems in the environment 100. For example, the healthcare provider computing device 104 is a computer system such as, for example, a desktop computer, a laptop computer, a tablet, a smart cellular phone, a smart watch, or other wearable computer, etc. The healthcare provider computing device 104 includes one or more applications, e.g., a program, plugin, browser extension, etc., installed on a memory of the respective computing device. In some embodiments, the applications are associated with one or more of the other components in the environment 100. For example, the applications include one or more of system control software, system monitoring software, software development tools, etc.

As shown in FIG. 1, at least a portion of one or more instructions stored in a memory of the healthcare provider computing device 104 is associated with a healthcare provider application 105 (abbreviated as HP application) that is configured to communicate with one or more of the server-side systems 106. For example, the healthcare provider application 105 is executed on the healthcare provider computing device 104 to communicate with the healthcare services system 108 to receive notifications when a patient has reported symptoms (e.g., via the automated patient interview performed and recorded by the patient interview system 110) and/or to retrieve the patient's electronic health record from one of the data storage system(s) 114. In some examples, the electronic health record includes a notation generated by the notation generation system 111 based on question and answer data from the automated patient interview. The healthcare provider application 105 is a similar application to the patient application 103 (e.g., hosted by a same provider of the healthcare services system 108), except that the healthcare provider application 105 specifically includes features of the healthcare services system 108 directed toward healthcare providers (e.g., as opposed to features directed toward patients).

In some examples, the healthcare provider application 105 is a thick client application that is installed locally on the healthcare provider computing device 104 (e.g., a desktop application or mobile application). In other examples, the healthcare provider application 105 is a thin client application (e.g., a web application) that is rendered via a web browser launched on the healthcare provider computing device 104.

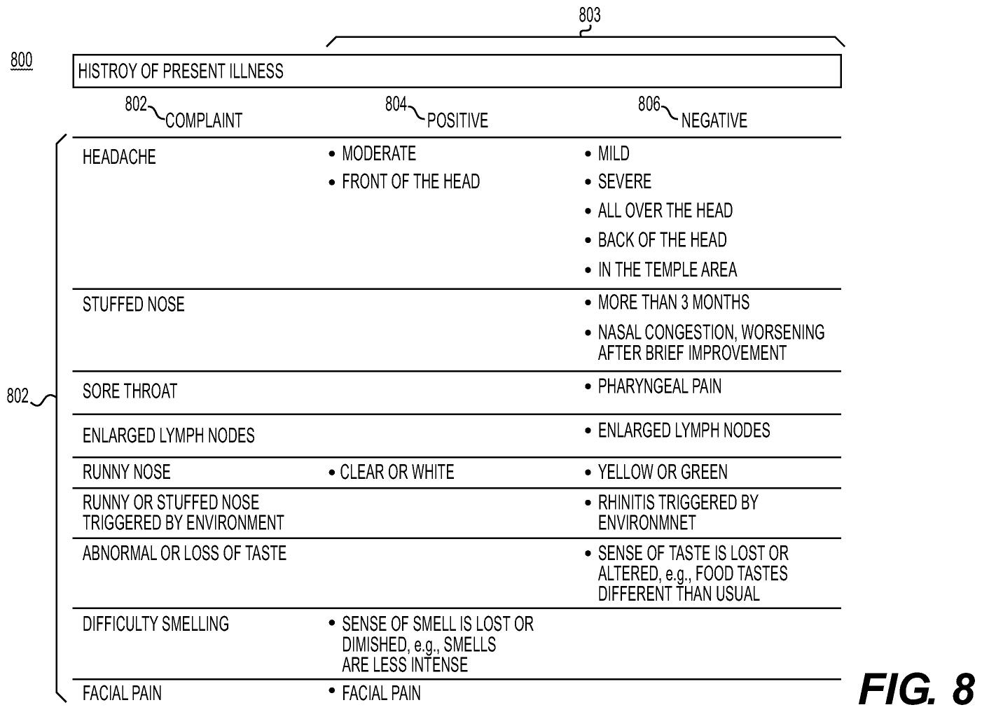

Additionally, one or more components of the healthcare provider computing device 104 generate, or cause to be generated, one or more graphic user interfaces (GUIs) based on instructions/information stored in the memory, instructions/information received from the other systems in the environment 100, and/or the like and cause the GUIs to be displayed via a display of the healthcare provider computing device 104. The GUIs can be, e.g., mobile application interfaces or browser user interfaces and include text, input text boxes, selection controls, and/or the like. In some examples, the display includes a touch screen or a display with other input systems (e.g., a mouse, keyboard, etc.) for the healthcare provider to control the functions of the healthcare provider computing device 104. An example GUI displayed via the display of the healthcare provider computing device 104 is shown in FIG. 8.

The healthcare services system 108 includes one or more server devices (or other similar computing devices) for executing healthcare services of a provider, such as a CDO. For example, through a session established via the patient application 103 executing on the patient computing device 102, the healthcare services system 108 enables the patient to access their electronic health records, as well as to report symptoms through an automated patient interview process and/or schedule a healthcare visit (e.g., to address the symptoms reported), among other activities. Additionally, through a session established via the healthcare provider application 105 executing on the healthcare provider computing device 104, the healthcare services system 108 enables the healthcare provider to receive notifications when a patient has reported symptoms, access electronic health records of the patient, and/or initiate scheduling of a healthcare visit for the patient, among other activities.

As described in detail elsewhere herein, the healthcare services system 108 includes an HPI object building tool 122 that is configured to receive question and answer data from an automated patient interview in a first format, and generate an HPI object to represent a portion of the question and answer data related to the patient's HPI (e.g., the HPI portion). The HPI object is further processed to convert the HPI portion into an easily readable, graphical format for inclusion in a notation. In some examples, the HPI object building tool 122 is configured to perform multi-threading to process questions from the question and answer data in parallel to increase a speed at which the HPI object building tool 122 generates the object.

The patient interview system 110 includes one or more server devices (or other similar computing devices) for executing automated patient interview services. As described elsewhere herein, example automated patient interview services include performing a patient interview that prompts the patient to provide answers to a series of presented questions, recording question and answer data from the patient interview, and processing the question and answer data to determine patient output data for provision as results of the patient interview, among other operations. The questions relate to symptoms the patient is experiencing, as well as patient medical history, and/or patient risk factors. The patient output data includes one or more possible causes, a recommended level of care, and/or a recommended care plan. For each of the possible causes, risk factors, symptoms, self-advice, and/or advice on when to see a healthcare provider, can also be displayed. Additionally or alternatively, the possible causes, recommended level of care, and/or recommended care plan are provided to the notation generation system 111 for inclusion in a notation.

The patient interview system 110 includes one or more tool(s) 124, including Al— or machine learning-based tools, for performing one or more of these automated patient interview services. For example, an AI-based application bot (e.g., provided via the patient application 103) performs and records the question and answer data from the patient interview. Additionally, one or more machine learning systems are implemented to determine the possible causes, recommended level of care, and/or recommended care plan based on the question and answer data. In some implementations, no personal health information (PHI) of the patient is provided to the patient interview system 110 (e.g., the question and answer data is de-identified) to help ensure legal compliance when the patient interview system 110 is hosted by a third party not privileged to PHI.

The notation generation system 111 includes one or more server devices (or other similar computing devices) for executing notation generation services. In some examples, the notation generation system 111 includes a database (e.g., AZURE COSMOS DB of Microsoft Corporation) for receiving and storing patient notation requests from one of the components of the environment 100 and a cron service for executing the processing of the patient notation requests at scheduled intervals. As described elsewhere herein, example notation generation services include generating a notation for inclusion in a patient's electronic health record. Data from the patient interview system 110 (e.g., the recorded question and answer data, the possible causes, the recommended level of care, and/or the recommended care plan) and the HPI object from the healthcare services system 108 are received and further processed by the notation generation system 111 to generate a notation.

The scheduling system 112 includes one or more server devices (or other similar computing devices) for executing scheduling services. As described elsewhere herein, example scheduling services include determining date and time availability for a healthcare provider to schedule a healthcare visit of a particular type (e.g., virtual visit vs. in-person visit, etc.), scheduling a date and time for the healthcare visit based on feedback from the patient and/or the healthcare provider, generating and providing a calendaring event to the patient and/or the healthcare provider, generating and transmitting notifications and/or reminders to the patient and/or the healthcare provider regarding the scheduled healthcare visit, etc. As one example, the scheduling system 112 receives a request for schedule availability of one or more healthcare providers and/or healthcare provider teams from the healthcare services system 108. In response to receipt of the request, the scheduling system 112 queries one or more data stores associated with the scheduling system 112, such as a scheduling data store 120 described in detail below, to determine the schedule availability. The scheduling system 112 transmits the determined schedule availability (e.g., available dates, times, etc.) to the healthcare services system 108 for display to the patient via the patient application 103.

The data storage system(s) 114 each include a server system or computer-readable memory such as a hard drive, flash drive, disk, etc. The data storage system(s) 114 include and/or act as a repository or source for various types of healthcare-related data. For example, the data storage system(s) 114 include a plurality of data stores, including a patient health data store 116, an HPI factors data store 118, and/or the scheduling data store 120, among other data stores. In some examples, one of the data storage system(s) 114 maintains each of the data stores 116, 118, 120. In other examples, one or more of the data stores 116, 118, and 120 are maintained across two or more different ones of the data storage system(s) 114.

The patient health data store 116 includes information associated with a plurality of patients, including electronic health records for the patients, contact information for the patients, healthcare providers that have previously seen the patients, patient insurance information, etc. A given patient's information is stored in the patient health data store 116 in association with a patient identifier. In some examples, the patient identifier is further associated with a patient's account with the healthcare services system 108. In such examples, when a patient logs into their account via the patient application 103, the patient identifier is identified and used to obtained information for that patient from the patient health data store 116.

The HPI factors data store 118 includes the parent-child symptom mapping referenced by the HPI object building tool 122 when processing the question and answer data to build the HPI object. The parent-child symptom mapping is a mapping of a plurality of parent symptoms to one or more child symptoms associated with each of the plurality of parent symptoms. The plurality of symptoms in the mapping include potential symptoms that could be indicated by the patient during the automated patient interview (e.g., symptoms recognizable by the patient interview system 110 when input and/or selected by the patient as part of the patient interview process). The one or more child symptoms for a parent symptom correspond to each of the answers that a patient can select in response to one or more questions asked about the parent symptom when the parent symptom is a complaint indicated by the patient during the patient interview. For example, when the patient indicates they have a cough, a first question may ask how long the cough has lasted, with answer options being 3 days or less, more than 3 days and less than a week, more than a week and less than two weeks, or more than two weeks. A second question may ask what the characteristics of the cough are, with the answer options being dry, wet, and wet and dry. In such an example, the parent symptom is cough and the associated child symptoms in the mapping may be each of the answer options from the first and second questions. In some examples, the mapping is generated by and/or periodically evaluated and updated by healthcare providers (e.g., physicians) to ensure the mapping includes a robust listing of parent and child symptoms.

In some examples, the HPI factors data store 118 is a non-relational database, such as a noSQL database, to facilitate management and/or updating of the information stored therein (e.g., of the mapping) by both technical administrators and non-technical administrators alike. For example, based on the non-relational structure of the HPI factors data store 118, each parent symptom in the mapping is stored in order with no defined relationships between the parent symptoms to provide scalability. However, in other examples, the HPI factors data store 118 is a graph database and/or relational database to depict relationships between parent symptoms. In such examples, natural language processing (NLP) (e.g., natural language understanding (NLU)) techniques are employed to group parent symptoms.

The scheduling data store 120 stores scheduling and availability information associated with one or more healthcare providers. For example, the scheduling and availability information for a healthcare provider is stored in association with an identifier for that healthcare provider. Accordingly, when a patient of the healthcare provider in conjunction with or separately from the patient interview process selects to schedule a healthcare visit with the provider via the patient application 103, the identifier of the healthcare provider can be used to obtain the scheduling and availability information from the scheduling data store 120.

The network 130 over which the one or more components of the environment 100 communicate includes one or more wired and/or wireless networks, such as a wide area network (“WAN”), a local area network (“LAN”), personal area network (“PAN”), a cellular network (e.g., a 3G network, a 4G network, a 5G network, etc.) or the like. In some embodiments, the network 130 includes the Internet, and information and data provided between various systems occurs online. “Online” means connecting to or accessing source data or information from a location remote from other devices or networks coupled to the Internet. Alternatively, “online” refers to connecting or accessing a network (wired or wireless) via a mobile communications network or device. The Internet is a worldwide system of computer networks—a network of networks in which a party at one computer or other device connected to the network can obtain information from any other computer and communicate with parties of other computers or devices. The most widely used part of the Internet is the World Wide Web (often-abbreviated “WWW” or called “the Web”). A “page” generally encompasses a location, data store, or the like that is, for example, hosted and/or operated by a computer system so as to be accessible online, and includes data configured to cause a program such as a web browser to perform operations such as send, receive, or process data, generate a visual display and/or an interactive interface, or the like. The patient computing device 102, the healthcare provider computing device 104, and one or more of the server-side systems 106 are connected via the network 130, using one or more standard communication protocols. The patient computing device 102, the healthcare provider computing device 104, and the one or more of the server-side systems 106 transmit and receive communications from each other across the network 130, as discussed in more detail below.

The various services or operations of the server-side systems 106 described above related to the patient interview process, HPI object building, notation generation, and/or automated scheduling are examples of serverless functions that are only performed (e.g., are only consuming processing resources of respective ones of the server-side systems 106) when triggered by a specific condition. An example specific trigger condition for the patient interview process includes an API call to initiate the patient interview process when the patient launches the symptom checker features via the patient application 103.

Although depicted as separate components in FIG. 1, it should be understood that a component or portion of a component in the system of the environment 100 is, in some embodiments, integrated with or incorporated into one or more other components. For example, the patient interview system 110, the notation generation system 111, the scheduling system 112 and/or the data storage system(s) 114 can be integrated with the healthcare services system 108 or the like. In some embodiments, operations or aspects of one or more of the components discussed above are distributed amongst one or more other components. Any suitable arrangement and/or integration of the various systems and devices of the environment 100 can be used.

In the following disclosure, various acts are described as performed or executed by a component from FIG. 1, such as the patient computing device 102, the healthcare provider computing device 104, or one or more of the server-side systems 106, or components thereof. However, it should be understood that in various aspects, various components of the environment 100 discussed above execute instructions or perform acts including the acts discussed below. An act performed by a device is considered to be performed by a processor, actuator, or the like associated with that device. Further, it should be understood that in various embodiments, various steps can be added, omitted, and/or rearranged in any suitable manner.

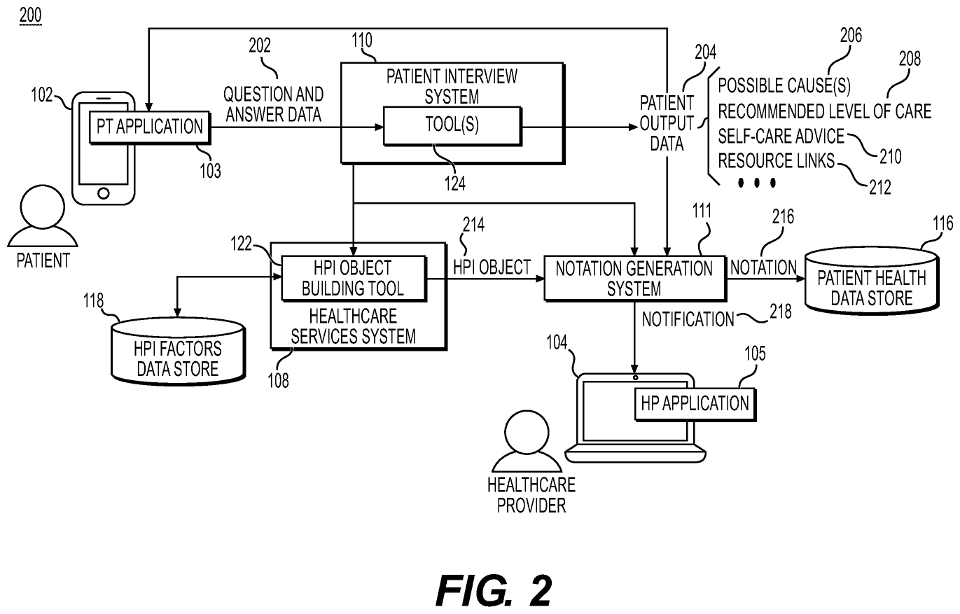

FIG. 2 is a system flow diagram 200 showing an example of a process for automatic health data processing, according to aspects of the disclosure. In some examples, the process is performed by one or components of the environment 100 described with reference to FIG. 1.

The patient application 103 that is executing on the patient computing device 102 prompts the patient to login using credentials, such as a username and password, for example. The patient application 103 establishes an application session upon a successful login, where the application session is associated with a patient identifier for the patient obtained based on the credentials provided at login (e.g., based on a query of the patient health data store 116 using the credentials). In response to the patient application 103 receiving input from the patient to launch a symptom checker feature associated with the patient interview system 110, an application bot (e.g., one of the tool(s) 124) of the patient interview system 110 is called to ask a series of questions to perform an automated patient interview. In some examples, the application bot is an AI-based bot. Each of the questions and answers are recorded with evidence to generate certain question and answer data 202. The question and answer data 202 is associated with a conversation identifier that is specific to the patient. In some examples, the conversation identifier is the patient identifier associated with the application session.

The questions asked are related to primary symptoms, secondary symptoms, medical history, risk factors, and/or the like. A portion of the questions may request text and/or speech input from the patient, such as questions related to the primary symptoms the patient is experiencing. Common names for symptoms are recognized (e.g., feeling sick vs. nausea). Additionally, a plurality of different languages are recognized and processed. When speech is provided as input, one or more of the tool(s) 124 of the patient interview system 110 uses natural language processing (NLP) and/or national language understanding (NLU) techniques to process the speech input. In further examples, the patient interview system 110 provides an option for the patient to upload an image via the patient application 103 to provide a visual representation of one or more of the symptoms. The tool(s) 124 of the patient interview system 110 are configured to process any uploaded images using image analysis techniques to identify the symptoms and/or characteristics associated with the symptoms.

Other questions request the patient to make a selection of one or more answer options provided to the patient. For example, for these types of questions, the questions asked include single answer questions, such as yes or no questions, where the patient can only select between one option or the other to answer. For example, the question may be “Have you had a flu vaccination within the past year?”, and the patient selects yes or no. The questions can also include group single options, where a plurality of answer options are provided to the user, however only one option is selectable. For example, the question may be “How strong is your headache?”, and the patient can select between mild, moderate, or strong. The questions can further include group multiple options, where a plurality of answer options are provided to the patient and more than one option is selectable by the patient to answer the question. For example, the question may be “Where is your chest pain located?”, and the patient can select one or more of between the shoulder blades, behind the breastbone, to the left upper limb, etc. As the patient is being asked and answering questions regarding their primary and/or secondary symptoms (e.g., secondary symptoms being related to the primary symptoms), deduplication logic is employed by the application bot to prevent duplicate questions from being presented the patient. Primary symptoms are symptoms directly reported by the patient during the interview process (e.g., I have a fever). In response to the primary symptoms reported, the patient interview system 110 determines other symptoms related to the primary symptom that may be useful in determining a diagnosis or treatment, for example, and the application bot presents questions related to the other symptoms. To provide an illustrative example, based on the primary symptom of a fever, a question asks if the patient is also experiencing a cough. If the patient responds positively (e.g., they are experiencing a cough), the patient interview system 110 determines the cough is a secondary symptom.

When recording the questions and answers with evidence to generate the question and answer data 202, each potential answer for every question asked is recorded along with evidence of “present” or “absent” based on whether the patient selected the answer. For example, if the question asked is the example group single question type of “How strong is your headache?”, for which the patient can select between mild, moderate, or strong, and the patient selects moderate, each of the following will be recorded for the question: (1) Mild with evidence of “absent”, moderate with evidence of “present”, and strong with evidence of “absent”. Additionally, for each potential answer, a question type (single, group single, group multiple) associated with the question and/or an identifier associated with the answer is recorded. An example portion of the question and answer data 202 in a first format recorded by the application bot is shown below:

```
[
  {
    “id”: [
      “s_2018”
    ],
    “question”: “How long has your chest pain lasted?”,
    “answer”: “Less than 30 minutes”,
    “type”: “group_single”,
    “evidence”: “present”
  },
  {
    “question”: “Where is your chest pain located and where does the pain move or spread to?”,
    “type”: “group_multiple”,
    “id”: [
      “s_51”
    ],
    “evidence”: “absent”,
    “answer”: “All over”
  },
  {
    “question”: “Where is your chest pain located and where does the pain move or spread to?”,
    “type”: “group_multiple”,
    “id”: [
      “s_1509”
    ],
    “evidence”: “absent”,
    “answer”: “Behind the breastbone”
  },
  {
    “question”: “Where is your chest pain located and where does the pain move or spread to?”,
    “type”: “group_multiple”,
    “id”: [
      ”s_2074”
    ],
    “evidence”: “absent”,
    “answer”: “To the left upper limb e.g., shoulder, arm, fingers”
  },
  {
    “question”: “Where is your chest pain located and where does the pain move or spread to?”,
    “type”: “group_multiple”,
    “id”: [
      “s_37”
    ],
    “evidence”: “absent”,
    “answer”: “Between the shoulder blades”
  },
  {
    “question”: “Where is your chest pain located and where does the pain move or spread to?”,
    “type”: “group_multiple”,
    “id”: [
      “s_36”
    ],
    “evidence”: “absent”,
    “answer”: “To the neck”
  }
].
```

The tool(s) 124 of the patient interview system 110 process the question and answer data 202 to generate certain patient output data 204. The patient output data 204 is provided to the patient as results of the patient interview via the patient application 103. For example, a first machine learning system (e.g., one of the tool(s) 124) receive, as input data, patient medical history, risk factors, and/or reported symptoms from the question and answer data 202. The first machine learning system processes the input data to predict, and provide as output, one or more possible causes 206 and a recommended level of care 208. For each of the possible causes 206, some self-care advice 210, including when to see a healthcare provider, and/or one or more resource links 212 are provided. The resource links 212 can also include a link to, e.g., a virtual care team that provides images and/or information about the team.

In some examples, the recommended level of care 208 is presented along with other available care options, and the patient is prompted to select which option they will seek. The patient's selection is recorded as part of the patient interview, along with the patient output data 204. In some examples, based on the recommended level of care 208 and/or the patient's selection (e.g., if the care level is of higher acuity), the patient interview system 110 initiates a direct communication (e.g., via messaging) between one or more healthcare providers of the patient, the patient, and/or a family member of the patient.

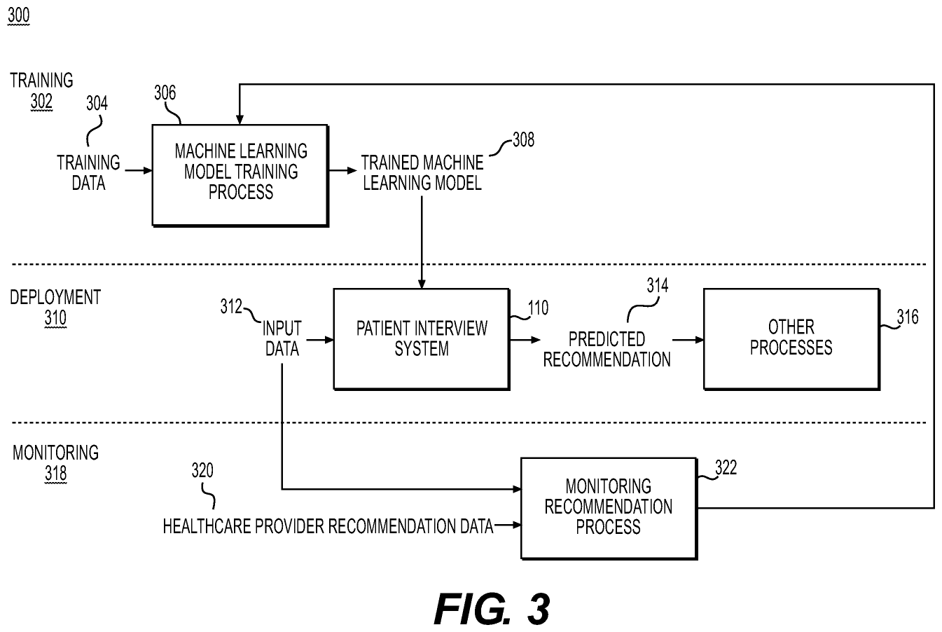

In some examples, and as described in more detail with reference to FIG. 3, the first machine learning system is further trained to predict, and provide as additional output, a recommendation associated with a care plan (e.g., a recommended diagnostic procedure, referral to specialist, etc.) based on the question and answer data 202. Alternatively, a second machine learning system different from the first machine learning system is trained to predict the diagnostic procedure. In some examples, the care plan-related recommendation is only provided to the healthcare provider.

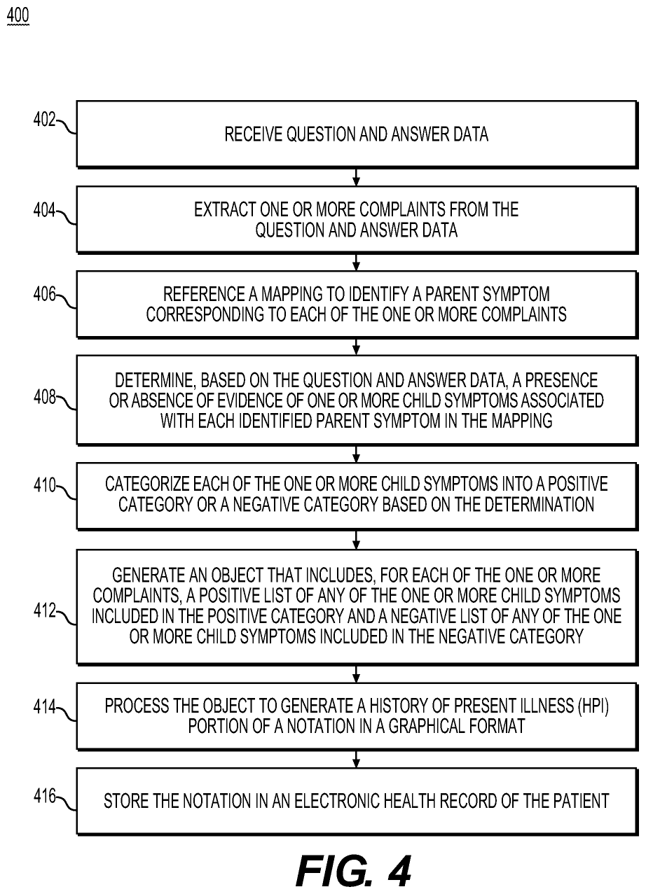

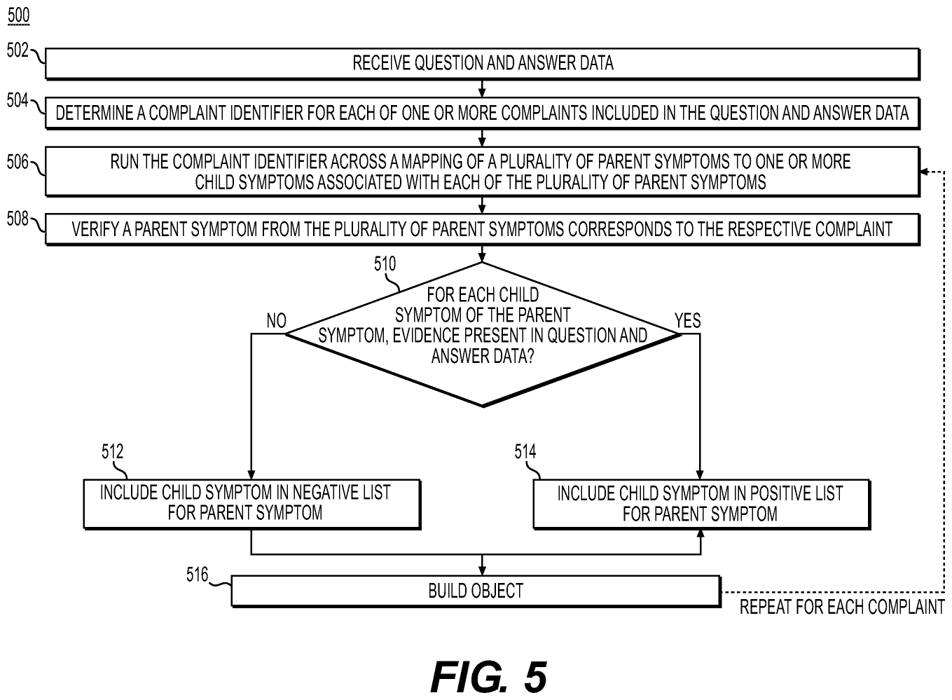

Additionally, the healthcare services system 108 receives the question and answer data 202, and provides the question and answer data 202 as input to the HPI object building tool 122. The HPI object building tool 122 processes the question and answer data 202 to automatically generate positive and negative lists for each HPI condition (e.g., complaint) extracted from the question and answer data in order to build a HPI object 214. To generate the positive and negative lists for each HPI condition, the parent-child symptom mapping stored in the HPI factors data store 118 is referenced. For example, and as described in more detail with reference to FIGS. 4 and 5 below, each HPI condition corresponds to a parent symptom from the mapping. A positive list for a given HPI condition includes each of the child symptoms associated with the parent symptom (e.g., the association determined from the mapping) for which the question and answer data 202 provides evidence that the patient is experiencing the child symptom. A negative list for a given HPI condition includes each of the child symptoms associated with the parent symptom for which the question and answer data 202 provides evidence that the patient is not experiencing the child symptom.

The HPI object 214 built by the HPI object building tool 122 is provided to the notation generation system 111 along with other patient interview-related data, such as the question and answer data 202, the patient output data 204, patient selections associated with the patient output data 204, and/or scheduling information (e.g., if a visit is automatically scheduled), for further processing. In some examples, the HPI object 214 and other patient interview related data is provided in a form of a patient notation request. In some examples, the healthcare services system 108 receives the patient interview-related data from the patient interview system 110 and/or scheduling system 112, and provides the patient interview-related data and the HPI object 214 in a single patient notation request to the notation generation system 111. In other examples, the patient interview-related data and the HPI object 214 are provided separately from the respective systems in two or more patient notation requests, wherein each request is associated with a patient identifier to enable the notation generation system 111 to group the requests for processing.

The notation generation system 111 processes the patient notation request(s) to generate a notation 216. In some examples, the cron service associated with the notation generation system 111 processes patient note requests at predefined intervals (e.g., every two to fifteen minutes). The notation 216 is in a form of a SOAP note. The HPI portion of the notation 216 (e.g., the portion based on the HPI object 214) is generated in a graphical format. In other words, the HPI portion of the question and answer data 202 from the first format recorded by the application bot is converted to the graphical format via the building and further processing of the HPI object 214. In some examples, the graphical format is a chart, table, or other similar tabular format where the rows represent the complaints extracted from the question and answer data, a first column represents a list of the positive intents for each complaint (e.g., a positive list), and a second column represents a list of the negative intents for each complaint (e.g., a negative list). An example HPI portion of the notation 216 is shown in FIG. 8. Although the present disclosure discusses charts and tables as examples of the graphical format to which the HPI portion is converted, the HPI portion can be converted to any suitable format that aids the viewing user (e.g., healthcare providers, patients, etc.) to easily discern and understand the patient's medical complaint(s), associated symptoms, and absence/presence of those symptoms. In other words, it should be understood that a graphical format is not limited to a chart, a table, and the like, and also includes any other suitable, human-readable format that aids the viewing user (e.g., healthcare providers, patients, etc.) to easily discern and understand the patient's medical complaint(s), associated symptoms, and absence/presence of those symptoms.

The graphical format of the HPI portion of the notation 216 is significantly easier and quicker for the healthcare provider team to read and comprehend the patient's symptoms as compared to reading an entirety of a transcript of the question and answer data 202 and manually building a HPI based on the transcript. For example, relying on just the transcript, the healthcare provider team would have to analyze the question and answer data 202 to identify parent and child symptoms and correlate them with the complaints of the patient to build and understand the patient's HPI. Therefore, by automatically identifying and analyzing parent and child symptoms using the parent-child symptom mapping and the question and answer data 202, and attaching the symptoms to the complaints of the patient as either positive (e.g., present) or negative (e.g., absent), the techniques described herein overcome challenges presented by the conventional, manual HPI building to facilitate analysis and decision making by the healthcare provider. Additionally, through the use of the parent-child symptom mapping, the HPI object building tool 122 prevents the repeated attachment of a same child symptom (e.g., a same response to a question associated with a parent symptom) to multiple body parts and/or parent symptoms.

The notation 216 is automatically stored in the patient's electronic health record in the patient health data store 116. For example, the conversation identifier associated with the question and answer data 202, which may correspond to the patient identifier as described above, is used to identify the patient's electronic health record for storage of the notation 216.

Additionally, once the notation 216 has been generated and stored, the notation generation system 111 generates and transmits a notification 218 to one or more members of the healthcare provider team to notify the team that the patient has reported symptoms. For example, the notification 218 can include one or more indications of an occurrence of the automated interview and availability of the notation 216 in the electronic health record for access. Example indications can include a parameter value, a flag, etc. In some examples, the notification 218 is sent via the healthcare provider application 105 executing on the healthcare provider computing device 104 (e.g., as a push notification). In other examples, the notification 218 is sent as an electronic communication, text message, instant message, etc. In further examples, the notification 218 is flagged or otherwise emphasized if the recommended level of care 208 determined is a higher acuity level of care. When multiple notifications are received at a given time, this allows the healthcare provider team to triage the patients to address those having more serious symptoms first, and appropriately allocate team resources.

Upon receipt of the notification 218, the healthcare provider application 105 is used by the healthcare provider to retrieve the notation 216 by requesting access to the patient's electronic health record that includes the notation 216. For example, the healthcare services system 108 receives the access request from the healthcare provider application 105, obtain the patient's electronic health record from the patient health data store 116 (e.g., using the patient identifier included in the access request), and transmit the patient's electronic health record to the healthcare provider application 105 in response to the access request.

Once retrieved, the healthcare provider may initiate a process of calling the patient, scheduling an appointment (if not already scheduled by the patient via the patient application), verifying the information, diagnosing, and/or treating the patient. The inclusion of the notation 216 in the electronic health record saves a healthcare provider team three notes per encounter, on average. For example, instead of multiple team members (e.g., an intake technician, a nurse practitioner, a physician, etc.) each having to essentially run through and notate patient answers to the same or similar questions asked during the automated patient interview, each team member may review the notation 216 prior to the patient interaction and/or correct or add to the notation 216 during the patient interaction. In an example scenario, if the patient is seen virtually or comes in for an in-person appointment, the physician may utilize the healthcare provider application 105 to copy and paste the notation 216 into a notes section for the visit that is documented in the patient's electronic health record. Any additional notation received as input from the physician to the healthcare provider application 105 to indicate corrections or adjustments to information included in the notation 216 or further details provided during the visit are captured and saved to the patient's electronic health record. In some examples, the healthcare provider may determine that they can diagnose and treat the patient's condition without talking live, real-time with the patient. Additionally or alternatively, the healthcare provider application 105 sends communications to the patient application 103 with additional questions or with a recommendation from the physician to escalate their level of care (e.g., from the virtual visit recommended by the symptom checker feature to in-person care).

FIG. 3 is a block diagram showing an example of a process 300 for training and using a machine learning model to output a healthcare-related recommendation based on patient interview data, according to certain embodiments. In some embodiments, the patient interview system 110 one or more of generates, stores, trains, or uses a machine learning model configured to determine a recommendation associated with a care plan for the patient based on at least the question and answer data 202 recorded during the patient interview. The patient interview system 110 includes a machine learning model and/or instructions associated with the machine learning model, e.g., instructions for generating a machine learning model, training the machine learning model, using the machine learning model, etc. In other embodiments, a system or device other than the patient interview system 110 is used to generate and/or train the machine learning model. For example, such a system includes instructions for generating the machine learning model and the training data, and/or instructions for training the machine learning model. A resulting trained-machine learning model is then provided to the patient interview system 110 for use.

As depicted in FIG. 3, in some examples, the process 300 includes a training phase 302, a deployment phase 310, and a monitoring phase 318. In the training phase 302, at step 306, the process 300 includes receiving and processing certain training data 304 to generate (e.g., build) a trained machine learning model 308 for predicting a recommendation associated with a patient's care plan, such as a recommended diagnostic procedure, specialist referral, etc.

The training data 304 includes a plurality of patient datasets. In some examples, a given patient dataset includes medical history information, risk factors, and symptoms reported by the patient. In some examples, the information included in the patient dataset is obtained and recorded during a previous automated patient interview performed using the patient interview system 110 (e.g., the training data 304 includes the question and answer data 202 from the previous automated patient interview). Additionally or alternatively, the information included in the patient dataset is obtained and recorded by a healthcare provider team member during a previous healthcare visit. The training data 304 is generated, received, or otherwise obtained from internal and/or external resources. For example, the training data 304 includes patient datasets collected from healthcare providers that are subscribers of the healthcare services provided by the healthcare services system 108. Additionally, or alternatively, the training data 304 includes patient datasets obtained from a third party and/or a public database.

Generally, a model includes a set of variables, e.g., nodes, neurons, filters, etc., that are tuned, e.g., weighted or biased, to different values via the application of the training data 304. In some examples, the training process at step 306 employs supervised, unsupervised, semi-supervised, and/or reinforcement learning processes to train the model (e.g., to result in the trained machine learning model 308). In some embodiments, a portion of the training data 304 is withheld during training and/or used to validate the trained machine learning model 308.

When supervised learning processes are employed, labels or scores facilitate the learning process by providing a ground truth. For example, the labels or scores indicate the actual care plan that was recommended by the patient's healthcare provider, including any diagnostic procedures (imaging, labs, diagnostic tests, etc.), specialist referrals, and/or the like. Training proceeds by feeding medical history information, risk factors, and symptoms reported (e.g., a sample) from a patient dataset of the training data into the model, the model having variables set at initialized values, e.g., at random, based on Gaussian noise, a pre-trained model, or the like. The model outputs a predicted recommendation for a care plan for the sample. The output is compared with the corresponding label or score (e.g., the ground truth) to determine an error, which is then back-propagated through the model to adjust the values of the variables. This process is repeated for a plurality of samples (e.g., a plurality of the patient datasets) at least until a determined loss or error is below a predefined threshold. In some examples, some of the training data 304 is withheld and used to further validate or test the trained machine learning model 308.

For unsupervised learning processes, the training data 304 is not include pre-assigned labels or scores to aid the learning process. Rather, unsupervised learning processes include clustering, classification, or the like to identify naturally occurring patterns in the training data 304. Supervised or unsupervised K-means clustering or K-Nearest Neighbors can also be used. Combinations of K-Nearest Neighbors and an unsupervised cluster technique can also be used. For semi-supervised learning, a combination of the training data 304 with pre-assigned labels or scores and the training data 304 without pre-assigned labels or scores are used to train the model.

When reinforcement learning is employed, an agent (e.g., an algorithm) is trained to make a decision regarding the care plan for the sample from the training data 304 through trial and error. For example, upon making a decision, the agent then receives feedback (e.g., a positive reward if the predicted recommendation was a recommendation that the healthcare provider agreed with and/or recommended), adjust its next decision to maximize the reward, and repeat until a loss function is optimized.

Once trained, the trained machine learning model 308 is stored and subsequently executed by one of the server-side systems 106, such as the patient interview system 110, during the deployment phase 310. For example, during the deployment phase 310, the trained machine learning model 308 receives certain input data 312 for a patient that has launched the symptom checker feature associated with the patient interview system 110 to report symptoms. The input data 312 includes medical history information, risk factors, and symptoms obtained from the question and answer data 202 of the automated patient interview. In some examples, additional information is obtained from the patient's electronic health record for inclusion in the input data 312. The trained machine learning model 308 outputs a predicted recommendation 314 associated with a care plan for the patient. The predicted recommendation 314 is then transmitted to one or more other ones of the server-side systems 106 for use in one or more other processes 316. For example, the predicted recommendation 314 is provided to the notation generation system 111 (e.g., as part of the patient notation request) for inclusion in the notation 216 viewable by the healthcare provider. In another example, if the predicted recommendation 314 includes a diagnostic procedure and/or a specialist referral, the predicted recommendation 314 is provided to the scheduling system 112 to determine a next availability to perform the procedure and/or a next availability of the specialist to see the patient.

During the monitoring phase 318, an actual care plan recommended by the healthcare provider (e.g., certain healthcare provider recommendation data 320) is collected. During process 322, the healthcare provider recommendation data 320 is analyzed along with the predicted recommendation 314 to determine an accuracy of the predicted recommendation 314. In some examples, based on the analysis, the process 300 returns to the training phase 302, where at step 306 values of one or more variables of the model are adjusted.

The process 300 described above is provided merely as an example, and can include additional, fewer, different, or differently arranged aspects than depicted in FIG. 3.

FIG. 4 is a flowchart showing an example of a process 400 for generating the notation 216, according to aspects of the disclosure. In some examples, the process 400 is performed by one or more of the server-side systems 106, such as the healthcare services system 108 and/or the notation generation system 111.

At step 402, the question and answer data 202 from an automated interview of a patient is received. For example, the question and answer data 202 is received as input data to the HPI object building tool 122 executed by the healthcare services system 108. At step 404, one or more complaints are extracted from the question and answer data 202. In some examples, the complaints are extracted using NLP processing techniques. The complaints correspond to symptoms reported by the patient. The symptoms include the initial set of one or more primary symptoms reported by the patient (e.g., in response to the question prompting the patient to add symptoms shown in FIG. 6D below) and/or any related secondary symptoms reported as a result of follow-up questions (e.g., in response to questions shown in FIGS. 6H and 61 below).

At step 406, a mapping is referenced to identify a parent symptom corresponding to each of the one or more complaints extracted at step 404. The mapping is the parent-child symptom mapping stored in the HPI factors data store 118 that includes a plurality of parent symptoms and one or more child symptoms associated with each of the plurality of parent symptoms. As described above in detail, the parent symptoms in the mapping includes at least each of the symptoms that is recognizable by the patient interview system 110 when input and/or selected by the patient as part of the patient interview process. The one or more child symptoms for a given parent symptom correspond to each of the answers that a patient can select in response to one or more questions asked about the parent symptom when the parent symptom is a complaint indicated by the patient during the patient interview. The parent symptom identified for each complaint is one of the plurality of parent symptoms within the mapping.

At step 408, a presence or absence of evidence of the one or more child symptoms associated with each identified parent symptom in the mapping is determined based on the question and answer data 202. As previously described, when the questions and answers are recorded with evidence to generate the question and answer data 202, each potential answer for every question asked is recorded along with evidence of “present” or “absent” based on whether the patient selected the answer. Therefore, for the subset of questions asked about each identified parent symptom, the answer(s) are recorded with evidence of “present” if the patient selected the answer or evidence of “absent” if the patient did not select the answer(s). Accordingly, the child symptoms of each identified parent symptom corresponding to the potential answers to the subset of questions impute the evidence recorded.

At step 410, each of the one or more child symptoms are categorized into a positive category or a negative category based on the determination. For example, if there is a presence of evidence of a child symptom, the child symptom is categorized into the positive category. Otherwise, if there is an absence of evidence of the child symptom, the child symptom is categorized into the negative category.

At step 412, an object (e.g., the HPI object 214) is generated that includes, for each of the one or more complaints, a positive list of any of the one or more child symptoms included in the positive category and a negative list of any of the one or more child symptoms included in the negative category. The HPI object 214 is then provided to the notation generation system 111 for further processing. For example, at step 414, the HPI object 214 is processed to generate an HPI portion of a notation for the patient in a graphical format.

The graphical format includes a chart, table, diagram or other similar graphical component having a tabular form. For example, the graphical format includes one or more rows representing the complaints, a first column representing the positive lists for the complaints, and a second column representing the negative lists for the complaints. Accordingly, within the graphical format, a first cell at the intersection of a first row representing a first complaint and the first column includes any of the child symptoms of the parent symptom corresponding to the first complaint for which evidence was present in the question and answer data 202. A second cell at the intersection of the first row and the second column includes any of the child symptoms of the parent symptom corresponding to the first complaint for which evidence was absent in the question and answer data 202.

In some examples, the HPI portion is just one portion or section of the notation 216. In such examples, other portions or sections of the notation 216 include portions listing chief complaints (e.g., indicating the primary and secondary symptoms reported), risk factors, and/or patient comments extracted from the question and answer data 202. Further portions or sections of the notation 216 include the patient output data 204 that was provided to the patient as results of the patient interview, such as the possible causes 206, the recommended level of care 208, and/or the self-care advice 210 and/or the resource links 212 provided for each of the possible causes, as well as a level of care selected by the patient responsive to the patient output data. Additionally, if the patient scheduled a healthcare visit, the scheduled date and time for the healthcare visit is included. Further, a transcript of the patient interview (e.g., in a format of the question asked and the answer selected by the patient) is generated and included in the notation 216.

In further examples, one or more natural language generation (NLG) processing techniques are applied to the question and answer data 202 to generate a summary format of the question and answer data 202 that is included in the notation 216. The summary format is a natural language output (e.g., a human readable output) that concisely summarizes at least a portion of the question and answer data 202, such as at least the portions of the questions and answer data 202 associated with the patient's HPI.

Example NLG processing includes extracting text phrases or utterances from the question and answer data 202, and providing the extracted text phrases or utterances as inputs to a classification system (e.g., including one or more machine learning models) trained to identify intents and entities. For example, the classification system identifies and outputs an intent for each of the text phrases or utterances and one or more entities corresponding to the intent. Intents include the patient's goal or purpose of providing a particular answer of the patient and answer data 202 as part of the automated patient interview (e.g., what the patient means), and the entities are semantic organizational data points that provide context or further describe the corresponding intents. As one example, the intents are parent symptoms and the entities are child symptoms corresponding to the respective parent symptoms, where the child symptoms can be categorized into positive or negative categories (child symptoms present or absent, respectively) as part of the classification system.

The intents and entities output by the classification system are then further processed (e.g., using one or more machine learning models) to generate natural language output. In some examples, the natural language output conforms to a standard format for the healthcare community (e.g., the standard format including specific information, such as patient age and gender in addition to symptoms). One illustrative example of a natural language output includes:
25 year old male patient presents with a cold and temperature of 101 degrees. The patient did not complain of the feeling of malaise or chills at any time in the past 3 days. The patient reported these symptoms on Jan. 1, 2023. The patient reports no other symptoms or concerns at this time.

At step 416, the notation 216 is stored in the patient's electronic health record. The electronic health record is subsequently accessed by the healthcare provider, via the healthcare provider application 105, to view the notation 216.

Accordingly, certain aspects of this disclosure include generating the notation 216. The process 400 described above is provided merely as an example, and can include additional, fewer, different, or differently arranged steps than depicted in FIG. 4.

FIG. 5 is a flowchart showing an example of a process 500 for building the HPI object 214, according to aspects of the disclosure. In some examples, the process 500 is performed by the HPI object building tool 122 executed by the healthcare services system 108. Process 500 can be used to perform at least a portion of the process 400 (e.g., to perform steps 402-412).

At step 502, the HPI object building tool 122 receives the question and answer data 202 as input. At step 504, a complaint identifier is determined for each of the one or more complaints included in the question and answer data 202. In some examples, the complaint identifier is a value (e.g., an alphanumeric value) that the patient interview system 110 assigns to a complaint included in the question and answer data. At step 506, the complaint identifier is compared against a mapping of a plurality of parent symptoms to one or more child symptoms associated with each of the plurality of parent symptoms (e.g., the parent-child symptom mapping stored in the HPI factors data store 118). At step 508, a parent symptom from the mapping (e.g., one of the plurality of parent symptoms) is verified as corresponding to the respective complaint using the complaint identifier. For example, the parent-child symptom mapping associates complaint identifiers assigned by the patient interview system 110 to each of the parent symptoms included in the mapping. In some examples, the parent-child symptom mapping also associates identifiers assigned by the patient interview system 110 to each of the child symptoms associated with the parent symptoms in the mapping.

Upon verification of the parent symptom, at decision 510, a determination is made for each child symptom of the parent symptom (e.g., indicated by the mapping) whether evidence of the child symptom is present in the question and answer data 202. If at decision 510, evidence of the child symptom is not present (e.g., is absent), then the child symptom is included in a negative list for the parent symptom at step 512. An absence of evidence of the child symptom indicates that the patient is not currently experiencing that child symptom. Otherwise, if at decision 510, evidence of the child symptom is present, then the child symptom is included in the positive list for the parent symptom at step 514. A presence of evidence of the child symptom indicates that the patient is currently experiencing that child symptom. Following the determination of whether evidence of the child symptom is present for each of the one or more child symptoms associated with the parent symptom, the negative list and the positive list for the parent symptom are utilized to build an object (e.g., the HPI object 214) at step 516.

Steps 506 through 516 are then be repeated for each complaint included in the question and answer data 202. Resultantly, the object built at step 516 includes, for each parent symptom corresponding to one of the complaints included in the question and answer data 202, a positive list of any child symptoms having evidence present in the question and answer data 202 and a negative list of any child symptoms not having evidence present in the question and answer data 202. In some examples, the HPI object 214 is a JSON (JavaScript Object Notation) formatted HPI object. The HPI object 214 is then provided to the notation generation system 111 (e.g., as part of a patient note request) for further processing to generate the HPI portion of a notation for the patient.

In some examples, the performance of steps 506 through 516 for each complaint are performed in parallel. For example, the HPI object building tool 122 is configured to perform multi-threading to increase a speed at which the question and answer data 202 are processed for each complaint to generate the HPI object 214. In some examples, portions of the HPI object 214 for the complaints that are generated in parallel may be associated with one another using the above-described conversation identifier that the question and answer data 202 is associated with. Such association facilitates aggregation of the portions of the HPI object 214 for each of the complaints identified in the question and answer data 202 to generate an entirety of the HPI object 214. As described in detail above, the conversation identifier can be the patient identifier associated with the application session established by the patient application 103 through which the automated patient interview was performed.

Accordingly, certain aspects of this disclosure include building objects that represent HPIs for patients. The process 500 described above is provided merely as an example, and can include additional, fewer, different, or differently arranged steps than depicted in FIG. 5.

FIGS. 6A-60 show examples of multiple patient interview user interfaces 600-628, according to aspects of the disclosure. The patient interview user interfaces 600-628 are generated and displayed by the patient application 103 executing on the patient computing device 102. For example, in response to receiving login information of the patient, the patient application 103 establishes a session associated with the patient identifier of the patient (e.g., based on credentials used to log in). The patient application 103 launches the symptom checker feature associated with the patient interview system 110 based on input from the patient to begin the interview process. Upon launching, the patient identifier is provided to the healthcare services system 108 and used by the healthcare services system 108 and/or the patient interview system 110 to obtain information about the patient, including past and/or current healthcare provider(s), patient demographics (e.g., age, sex, etc.), and medical history and/or risk factors such as prior diagnoses, medications taken by the patient, previous lab results.

As shown in FIG. 6A, the first patient interview user interface 600 displayed by the patient application 103 includes information to inform the patient as to what the interview process will entail and prompts the patient to provide consent to the sharing of information for this encounter with the patient's healthcare provider (e.g., the current or most healthcare provider indicated in the patient information). The patient may alternatively select to share the information with one or more other or different healthcare providers. Upon providing consent, the second patient interview user interface 602 is displayed to the patient as shown in FIG. 6B. The second patient interview user interface 602 is a compliance-related page that clarifies the purpose of the symptom checker feature and requests that the patient agree to the terms of use and the privacy policy of the symptom checker feature.

Once the user agrees, the third patient interview user interface 604 is displayed to the patient as shown in FIG. 6C. The third patient interview user interface 604 requires the patient to indicate whether they are experiencing a life-threatening emergency. If the patient indicates that it is a life-threatening emergency, they are prompted to instead call 911 (and/or be directly transferred to 911) rather than to report symptoms. Additionally, the notation 216 generated based on the question and answer data from the interview indicates that no symptoms were reported. Otherwise, if the patient indicates that they are not experiencing a life-threatening emergency, then the patient is asked to proceed with reporting symptoms via the fourth patient interview user interface 606 shown in FIG. 6D.

The fourth patient interview user interface 606 includes a prompt 630 for the patient to provide input to a search box 632 and/or select an area of a representative human body 634 (e.g., homunculus) to report the symptoms they are experiencing (e.g., primary symptoms). In some examples, if an area of the representative human body 634 is selected, the patient application 103 automatically populates symptoms associated with the area for patient selection to indicate which symptoms the patient is experiencing. In other examples, when the patient application 103 receives patient input to the search box 632 to report the symptom (e.g., receives a first few words), the patient application 103 performs auto-suggestion and/or auto-completion of the input. Additionally or alternatively, when the patient application 103 receives patient input to the search box 632 to report the symptom, the patient application 103 highlights one or more areas of the body that may experience the symptom on the representative human body 634, and the patient selects which area(s) they are experiencing the symptom in.

Common names for symptoms are recognized (e.g., feeling sick vs. nausea). Additionally, a plurality of different languages are recognized and processed. In other examples, the patient may speak their symptoms, and one or more of the tool(s) 124 of the patient interview system 110 use natural language processing (NLP) and/or national language understanding (NLU) techniques to extract the reported symptoms. In further examples, the patient interview system 110 provides an option for the patient to upload an image via the patient application 103 to provide a visual representation of one or more of the symptoms reported.

As a symptom is input and/or selected, the symptom is displayed on the fourth patient interview user interface 606 (e.g., symptoms 636). Once the patient indicates that they have completed the reporting of the symptoms, the fifth patient interview user interface 608 is displayed as shown in FIG. 6E. The fifth patient interview user interface 608 prompts the patient to indicate which type of care (e.g., level of care) that patient is considering based on the symptoms. Example levels of care include self-care, virtual visit, in-person visit, urgent care visit, emergency room visit, or call 9-1-1, among other examples. The type or level selected by the patient is recorded and studied to better understand how patients understand and correlate their symptoms to different levels of care and how use of the symptom checker feature (e.g., the recommended level of care 208) affects what level of care the patient ultimately seeks.

In addition to reporting symptoms, the patient application 103 prompts the patient to provide medical history information and/or information regarding risk factors (e.g., asked questions about weight, activity level, tobacco use, etc.), where some of the questions are prompted based on symptoms entered. The questions are also based on the demographics obtained by the patient information. For example, if the patient is a female of reproductive age, the patient is asked if they are pregnant. As shown in FIG. 6F, the sixth patient interview user interface 610 prompts the patient to indicate any pre-existing conditions. As shown in FIG. 6G, the seventh patient interview user interface 612 lists conditions retrieved from the patient's medical history information. If the status of the patient's condition has changed (e.g., if the patient was previously obese and is now no longer obese), the patient is provided an option to deselect the condition from the list via the seventh patient interview user interface 612.

Based on the primary symptoms reported and/or the medical history information and risk factors provided, additional questions related to any potential secondary symptoms that the patient is experiencing and/or characteristics of one or more of the primary and/or secondary symptoms (e.g., how long the symptom has lasted, a type of pain associated with the symptom, a color, an odor, etc.) is presented by the patient application 103. As the patient is being asked and answering questions regarding the primary and/or symptoms, deduplication logic is employed by the patient interview system 110 (e.g., by one of the tool(s) 124) to prevent duplicate questions from being presented the patient.

Example questions related to potential secondary symptoms are shown in the eighth patient interview user interface 614 shown in FIG. 6H and the ninth patient interview user interface 616 shown in FIG. 6I. Example questions related to characteristics of one or more of the primary and/or secondary symptoms are shown in the tenth patient interview user interface 618 shown in FIG. 6J, the eleventh patient interview user interface 620 shown in FIG. 6K, and the twelfth patient interview user interface 624 shown in FIG. 6M. One or more of the patient interview user interfaces 600-628 presenting questions to the patients include an information icon 640 displayed in conjunction with the question, as shown in the eleventh patient interview user interface 620 in FIG. 6K. Upon selection of the information icon 640, a notification 642 is displayed on the eleventh patient interview user interface 620 that includes additional information about what the question means and/or is asking for as shown in FIG. 6L. Additionally, as shown in FIG. 6N, the thirteenth patient interview user interface 626, includes a comments field 644 to allow the patient to input any additional comments about their symptoms, treatments attempted, and/or any other information for the healthcare provider to know.

As described above in greater detail, one or more of the tool(s) 124 of the patient interview system 110 processes the question and answer data 202 collected during the patient interview to generate the patient output data 204, including the possible causes 206 and the recommended level of care 208, among other information, such as the self-care advice 210 or the resource links 212. As shown in the FIG. 6O, the fourteenth patient interview user interface 628 is a results page including one or more sections 650, 652, 654.

The first section 650 describes the recommended level of care 208. The second section 652 indicates the possible causes 206. Control elements included in the second section 652 enable each of the possible causes 206 to be expandable to show the risk factors and symptoms associated with the respective cause, along with the self-care advice 210 for treating the cause and/or advice on when to see a healthcare provider. The third section 654 presents the recommended level of care 208 (e.g., indicated as such) among other available levels of care, and the patient is prompted to select which level of care they will seek. In some examples, the patient is only prompted to select a level of care if the recommended option is an elevated option (e.g., go to emergency room or call 9-1-1). In other examples, the patient is also presented with an option to directly message or contact the healthcare provider through the patient application 103 (e.g., via text, email, call, or video conferencing). Certain levels of care are restricted to a subset of patients (e.g., virtual visits are only available to patients of a minimum age). In such instance, the level of the care is only presented as an available option if the patient is eligible (e.g., meets the minimum age).

The prompts and/or control elements of the patient interview user interfaces 600-628 generated and displayed by the patient application 103 include standardized language to provide uniformity for the patient. The control elements are interacted with using a variety of input features, such as tab or enter in addition to selecting or clicking via a mouse, touch input, etc., to advance from one user interface page to the next. Additionally, in some examples, the user interface pages display a progress bar, and as the patient progresses through the interview process, the progress bar is updated to indicate a percentage of completion. Further, after completion, the patient is enabled to change or select additional providers to share the information from this encounter with (e.g., if select virtual visit, the patient updates to have the information from this symptom check encounter sent to the virtual care team). Additionally, the patient can be prompted to provide feedback on how to improve the patient experience.

The patient interview user interfaces 600-628 described above are provided merely as an example, and can include additional, fewer, different, or differently arranged information and/or interactive control elements than depicted in FIGS. 6A-60.

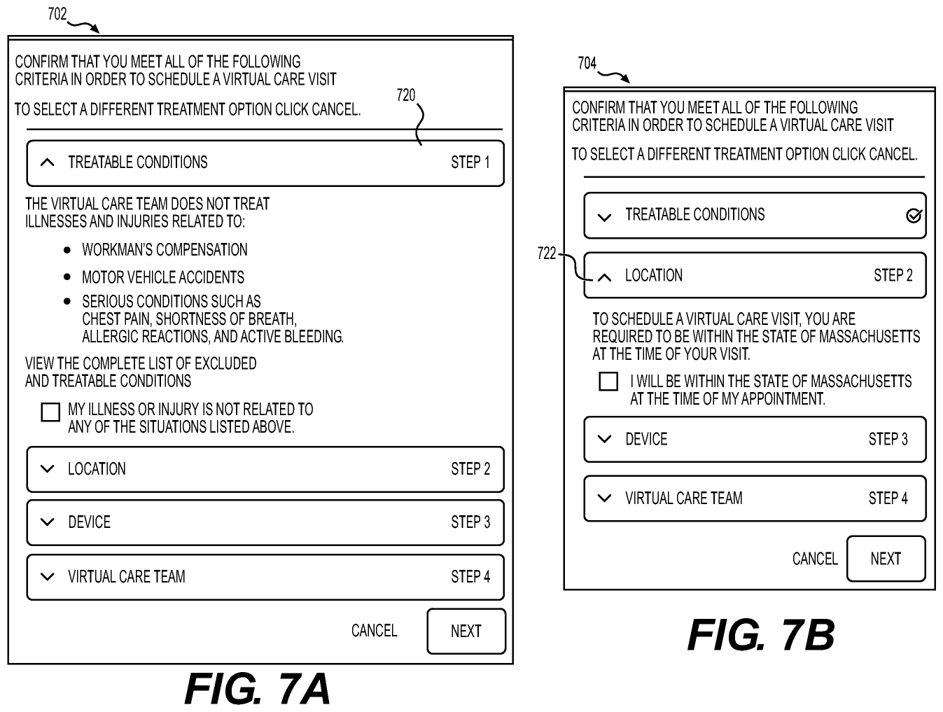

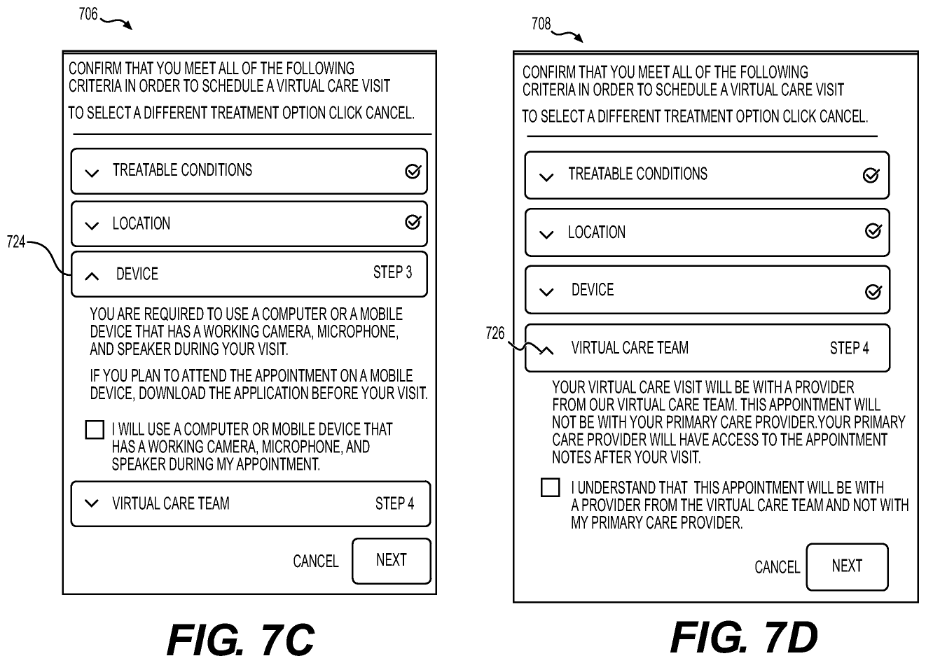

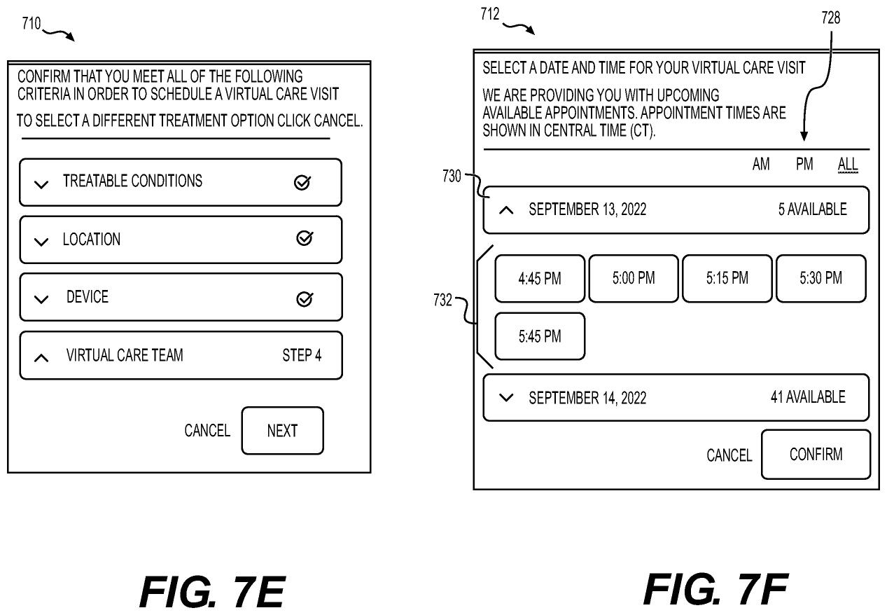

FIGS. 7A-7F show examples of multiple scheduling user interfaces 702-712, according to aspects of the disclosure. The scheduling user interfaces 702-712 are generated and displayed by the patient application 103 executing on the patient computing device 102. In some examples, based on the level of care selected by the patient (e.g., on the results user interface shown in FIG. 6O), the patient application 103 automatically directs the patient to schedule a healthcare visit corresponding to the level of care via a scheduling feature of the patient application 103 associated with the scheduling system 112. For example, if the patient selects to schedule a virtual care visit, automatic scheduling is provided to the patient. To be able to schedule a virtual care visit, various criteria associated with the patient's condition, location, device requirements, and/or virtual care team need to be met and/or acknowledged.

As shown in FIG. 7A, the first scheduling user interface 702 prompts the patient to verify that a first criteria 720 associated with the patient's condition is met. For example, the first criteria 720 is the condition is not related to any conditions that are not treatable via a virtual care visit. Upon verifying the first criteria 720 is met, the second scheduling user interface 704 is generated and displayed that prompts the patient to verify that a second criteria 722 associated with the patient's location is met, as shown in FIG. 7B. For example, the second criteria 722 is that the patient will be in a given state at the time of the virtual visit. Upon verifying the second criteria 722 is met, the third scheduling user interface 706 is generated and displayed that prompts the patient to verify that a third criteria 724 associated with the device requirements for the virtual care visit will be met, as shown in FIG. 7C. For example, the patient is required to use a device that has a camera, microphone, and speaker. Additionally, the patient may have to download a conferencing application to be used for the virtual care visit. Upon verifying the third criteria 724 is met, the fourth scheduling user interface 708 is generated and displayed that prompts the patient to acknowledge a fourth criteria 726 associated with the virtual care team, as shown in FIG. 7D. For example, the patient is prompted to acknowledge their understanding that the virtual care visit will not be with the patient's primary healthcare provider but with a virtual care team member.

Once each of the criteria has been indicated as being met and/or acknowledged, as shown in the fifth scheduling user interface 710 of FIG. 7E, the virtual care visit can be scheduled. For example, as shown in FIG. 7F, the sixth scheduling user interface 712 is generated and displayed that enables the user to select a time of day preference 728 (e.g., am, pm, or either) for the virtual care visit. For each day 730 having appointments available, one or more available appointment times 732 corresponding to the time of day preference 728 are displayable. The patient can select one of the available appointment times 732 on a given day 730 to schedule the appointment.

The scheduling user interfaces 702-712 described above are provided merely as an example, and can include additional, fewer, different, or differently arranged information and/or interactive control elements than depicted in FIGS. 7A-7F.

FIG. 8 shows an example of an HPI portion 800 of the notation 216, according to aspects of the disclosure. As shown, the HPI portion 800 is presented in a graphical format. The graphical format includes a chart, table, diagram, or other similar graphical component having a tabular form.

The graphical format includes one or more rows 802 and multiple columns 803. The rows 802 represent each of the complaints extracted from the question and answer data 202. The complaints include primary complaints initially reported by the patient and/or related secondary complaints determined based on answers to additional questions presented during the patient interview process. The columns 803 include a first column 804 representing the positive list for the complaints, and a second column 806 representing the negative list for the complaints.

The positive list for a complaint includes any child symptoms of a parent symptom that corresponds to the complaint for which there is a presence of evidence in the question and answer data 202. That is, any child symptom the patient indicates they are experiencing based on their answer to a question related to the parent symptom is included in the positive list. Accordingly, as depicted in FIG. 8, a first cell at the intersection of a first row (of the rows 802) representing a complaint of headache and the first column 804 includes the answers selected by the patient to questions regarding the strength of their headache (e.g., moderate) and what area of the head they were experiencing the headache in (e.g., front of the head).

The negative list for a complaint includes any child symptoms of a parent symptom that corresponds to the complaint for which there is an absence of evidence in the question and answer data 202. That is, any child symptom that the patient does not indicate they are experiencing based on their non-selection of an answer to a question related to the parent symptom is included in the negative list. Accordingly, as depicted in FIG. 8, a second cell at the intersection of the first row (of the rows 802) representing the complaint of headache and the second column 806 includes the answers to the questions regarding the strength of their headache and what area of the head they were experiencing the headache in that the patient did not select.

In some examples, one or more additional ones of the columns 803 are included, such as a column indicating an ambiguous or unknown intent (e.g., if a patient answers “I don't know” to a question).

The HPI portion 800 described above is provided merely as an example, and can include additional, fewer, different, or differently arranged information and/or be in a different graphical format than depicted in FIG. 8.

In general, any process or operation discussed in this disclosure that is understood to be computer-implementable can be performed by one or more processors of a computer system as described herein. A process or process step performed by one or more processors is also referred to as an operation. The one or more processors are configured to perform such processes by having access to instructions (e.g., software or computer-readable code) that, when executed by the one or more processors, cause the one or more processors to perform the processes. The instructions are stored in a memory of the computer system. A processor can be a central processing unit (CPU), a graphics processing unit (GPU), or any suitable types of processing unit.

A computer system, such as a system or device implementing a process or operation in the examples above, includes one or more computing devices. One or more processors of a computer system can be included in a single computing device or distributed among a plurality of computing devices. One or more processors of a computer system can be connected to a data storage device. A memory of the computer system includes the respective memory of each computing device of the plurality of computing devices.

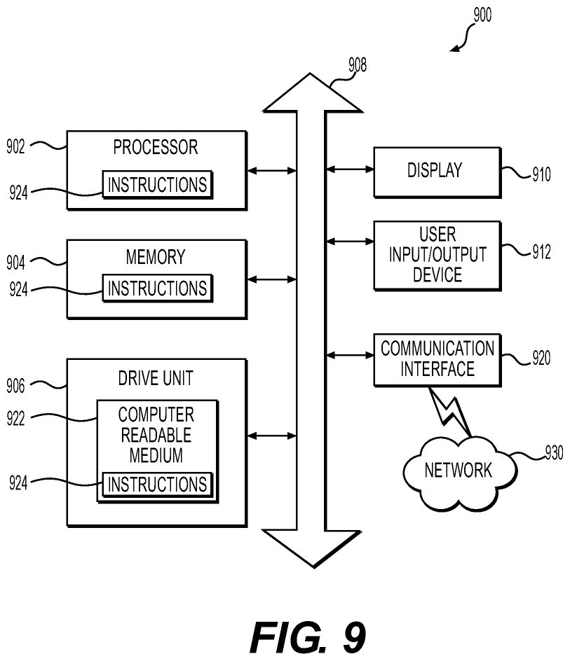

FIG. 9 illustrates an implementation of a computer system 900 that executes techniques presented herein. The computer system 900 can include a set of instructions that can be executed to cause the computer system 900 to perform any one or more of the methods or computer-based functions disclosed herein. The computer system 900 operates as a standalone device or is connected, e.g., using a network, to other computer systems or peripheral devices.

Unless specifically stated otherwise, as apparent from the following discussions, it is appreciated that throughout the specification, discussions utilizing terms such as “processing,” “computing,” “calculating,” “determining”, analyzing” or the like, refer to the action and/or processes of a computer or computing system, or similar electronic computing device, that manipulate and/or transform data represented as physical, such as electronic, quantities into other data similarly represented as physical quantities.

In a similar manner, the term “processor” refers to any device or portion of a device that processes electronic data, e.g., from registers and/or memory to transform that electronic data into other electronic data that, e.g., is stored in registers and/or memory. A “computer,” a “computing machine,” a “computing platform,” a “computing device,” or a “server” includes one or more processors.

In a networked deployment, the computer system 900 operates in the capacity of a server or as a client user computer in a server-client user network environment, or as a peer computer system in a peer-to-peer (or distributed) network environment. The computer system 900 can also be implemented as or incorporated into various devices, such as a personal computer (PC), a tablet PC, a set-top box (STB), a personal digital assistant (PDA), a mobile device, a palmtop computer, a laptop computer, a desktop computer, a communications device, a wireless telephone, a land-line telephone, a control system, a camera, a scanner, a facsimile machine, a printer, a pager, a personal trusted device, a web appliance, a network router, switch or bridge, or any other machine capable of executing a set of instructions (sequential or otherwise) that specify actions to be taken by that machine. In a particular implementation, the computer system 900 can be implemented using electronic devices that provide voice, video, or data communication. Further, while the computer system 900 is illustrated as a single system, the term “system” shall also be taken to include any collection of systems or sub-systems that individually or jointly execute a set, or multiple sets, of instructions to perform one or more computer functions.

As illustrated in FIG. 9, the computer system 900 includes a processor 902, e.g., a central processing unit (CPU), a graphics processing unit (GPU), or both. The processor 902 can be a component in a variety of systems. For example, the processor 902 is part of a standard personal computer or a workstation. The processor 902 is one or more processors, digital signal processors, application specific integrated circuits, field programmable gate arrays, servers, networks, digital circuits, analog circuits, combinations thereof, or other now known or later developed devices for analyzing and processing data. The processor 902 implements a software program, such as code generated manually (e.g., programmed).

The computer system 900 includes a memory 904 that can communicate via a bus 908. The memory 904 is a main memory, a static memory, or a dynamic memory. The memory 904 includes, but is not limited to computer readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media, and the like. In one implementation, the memory 904 includes a cache or random-access memory for the processor 902. In alternative implementations, the memory 904 is separate from the processor 902, such as a cache memory of a processor, the system memory, or other memory. The memory 904 can be an external storage device or database for storing data. Examples include a hard drive, compact disc (“CD”), digital video disc (“DVD”), memory card, memory stick, floppy disc, universal serial bus (“USB”) memory device, or any other device operative to store data. The memory 904 is operable to store instructions executable by the processor 902. The functions, acts or tasks illustrated in the figures or described herein are performed by the processor 902 executing the instructions stored in the memory 904. The functions, acts or tasks are independent of the particular type of instructions set, storage media, processor or processing strategy and are performed by software, hardware, integrated circuits, firm-ware, micro-code and the like, operating alone or in combination. Likewise, processing strategies can include multiprocessing, multitasking, parallel processing, and the like.

As shown, the computer system 900 further included a display 910, such as a liquid crystal display (LCD), an organic light emitting diode (OLED), a flat panel display, a solid-state display, a cathode ray tube (CRT), a projector, a printer or other now known or later developed display device for outputting determined information. The display 910 acts as an interface for the user to see the functioning of the processor 902, or specifically as an interface with the software stored in the memory 904 or in a drive unit 906.

Additionally or alternatively, the computer system 900 includes an input/output device 912 configured to allow a user to interact with any of the components of the computer system 900. The input/output device 912 is a number pad, a keyboard, or a cursor control device, such as a mouse, or a joystick, touch screen display, remote control, or any other device operative to interact with the computer system 900.

The computer system 900 also or alternatively includes the drive unit 906 implemented as a disk or optical drive. The drive unit 906 includes a computer-readable medium 922 in which one or more sets of instructions 924, e.g., software, can be embedded. Further, the sets of instructions 924 embody one or more of the methods or logic as described herein. The instructions 924 reside completely or partially within the memory 904 and/or within the processor 902 during execution by the computer system 900. The memory 904 and the processor 902 can also include computer-readable media as discussed above.

In some systems, the computer-readable medium 922 includes the sets of instructions 924 or receives and executes the sets of instructions 924 responsive to a propagated signal so that a device connected to a network 930 can communicate voice, video, audio, images, or any other data over the network 930. Further, the sets of instructions 924 are transmitted or received over the network 930 via a communication port or interface 920, and/or using the bus 908. The communication port or interface 920 is a part of the processor 902 or is a separate component. The communication port or interface 920 is created in software or is a physical connection in hardware. The communication port or interface 920 are configured to connect with the network 930, external media, the display 910, or any other components in the computer system 900, or combinations thereof. The connection with the network 930 is a physical connection, such as a wired Ethernet connection or is established wirelessly as discussed below. Likewise, the additional connections with other components of the computer system 900 are physical connections or are established wirelessly. The network 930 is alternatively directly connected to the bus 908.

While the computer-readable medium 922 is shown to be a single medium, the term “computer-readable medium” includes a single medium or multiple media, such as a centralized or distributed database, and/or associated caches and servers that store one or more sets of instructions. The term “computer-readable medium” also includes any medium that is capable of storing, encoding, or carrying a set of instructions for execution by a processor or that cause a computer system to perform any one or more of the methods or operations disclosed herein. In some examples, the computer-readable medium 922 is non-transitory, and is tangible.

The computer-readable medium 922 can include a solid-state memory such as a memory card or other package that houses one or more non-volatile read-only memories. The computer-readable medium 922 can be a random-access memory or other volatile re-writable memory. Additionally or alternatively, the computer-readable medium 922 can include a magneto-optical or optical medium, such as a disk or tapes or other storage device to capture carrier wave signals such as a signal communicated over a transmission medium. A digital file attachment to an e-mail or other self-contained information archive or set of archives are considered a distribution medium that is a tangible storage medium. Accordingly, the disclosure is considered to include any one or more of a computer-readable medium or a distribution medium and other equivalents and successor media, in which data or instructions are storable.

In an alternative implementation, dedicated hardware implementations, such as application specific integrated circuits, programmable logic arrays and other hardware devices, can be constructed to implement one or more of the methods described herein. Applications that include the apparatus and systems of various implementations can broadly include a variety of electronic and computer systems. One or more implementations described herein implement functions using two or more specific interconnected hardware modules or devices with related control and data signals that can be communicated between and through the modules, or as portions of an application-specific integrated circuit. Accordingly, the present system encompasses software, firmware, and hardware implementations.

The computer system 900 is connected to the network 930. The network 930 defines one or more networks including wired or wireless networks, such as the network 130 described in FIG. 1. The wireless network can be a cellular telephone network, an 802.11, 802.19, 802.20, or WiMAX network. Further, such networks include a public network, such as the Internet, a private network, such as an intranet, or combinations thereof, and utilize a variety of networking protocols now available or later developed including, but not limited to TCP/IP based networking protocols. The network 930 can include wide area networks (WAN), such as the Internet, local area networks (LAN), campus area networks, metropolitan area networks, a direct connection such as through a Universal Serial Bus (USB) port, or any other networks that allow for data communication. The network 930 is configured to couple one computing device to another computing device to enable communication of data between the devices. The network 930 generally is enabled to employ any form of machine-readable media for communicating information from one device to another. The network 930 includes communication methods by which information may travel between computing devices. The network 930 can be divided into sub-networks. The sub-networks allow access to all of the other components connected thereto or the sub-networks restrict access between the components. The network 930 can be regarded as a public or private network connection and can include, for example, a virtual private network or an encryption or other security mechanism employed over the public Internet, or the like.

In accordance with various implementations of the present disclosure, the methods described herein are implemented by software programs executable by a computer system. Further, in one example, non-limited implementation, implementations can include distributed processing, component/object distributed processing, and parallel processing. Alternatively, virtual computer system processing can be constructed to implement one or more of the methods or functionalities as described herein.

Although the present specification describes components and functions that are implemented in particular implementations with reference to particular standards and protocols, the disclosure is not limited to such standards and protocols. For example, standards for Internet and other packet switched network transmission (e.g., TCP/IP, UDP/IP, HTML, HTTP) represent examples of the state of the art. Such standards are periodically superseded by faster or more efficient equivalents having essentially the same functions. Accordingly, replacement standards and protocols having the same or similar functions as those disclosed herein are considered equivalents thereof.

It will be understood that the steps of methods discussed are performed in one embodiment by an appropriate processor (or processors) of a processing (e.g., computer) system executing instructions (computer-readable code) stored in storage. It will also be understood that the disclosure is not limited to any particular implementation or programming technique and that the disclosure is implementable using any appropriate techniques for implementing the functionality described herein. The disclosure is not limited to any particular programming language or operating system.

It should be appreciated that in the above description of example embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the invention can be practiced without these specific details. In other instances, well-known methods, structures, and techniques have not been shown in detail in order not to obscure an understanding of this description.

Thus, while there has been described what are believed to be the preferred embodiments of the invention, those skilled in the art will recognize that other and further modifications can be made thereto without departing from the spirit of the invention, and it is intended to claim all such changes and modifications as falling within the scope of the invention. For example, any formulas given above are merely representative of procedures that can be used. Functionality can be added or deleted from the block diagrams and operations are interchangeable among functional blocks. Steps can be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations and implementations are possible within the scope of the disclosure. Accordingly, the disclosure is not to be restricted except in light of the attached claims and their equivalents.
Example 1. A computer-implemented method comprising: receiving, by one or more processors, question and answer data from an automated interview of a patient; extracting, by the one or more processors, one or more complaints from the question and answer data; referencing, by the one or more processors, a mapping that includes a plurality of parent symptoms and a plurality of child symptoms associated with the plurality of parent symptoms, to identify a respective parent symptom from the plurality of parent symptoms corresponding to each of the one or more complaints; determining, by the one or more processors and based on the question and answer data, a presence or absence of evidence of one or more child symptoms, among the plurality of child symptoms, associated with each identified parent symptom; categorizing, by the one or more processors, each of the one or more child symptoms into a positive category or a negative category based on the determination; generating, by the one or more processors, an object that includes, for each of the one or more complaints, (a) a positive list including any of the one or more child symptoms included in the positive category and (b) a negative list including any of the one or more child symptoms included in the negative category; processing, by the one or more processors, the object to generate a history of present illness (HPI) portion of a notation for the patient in a graphical format; storing, by the one or more processors, the notation in an electronic health record of the patient; and generating and transmitting, by the one or more processors and to a healthcare provider application, a notification that includes one or more indications of an occurrence of the automated interview and availability of the notation in the electronic health record for access.
Example 2. The computer-implemented method of example 1, further comprising: processing, by the one or more processors, the question and answer data using one or more natural language generation (NLG) processing techniques to generate a summary format of the question and answer data, wherein the notation further includes the summary format.
Example 3. The computer-implemented method of example 1 or 2, further comprising: processing, by the one or more processors, the question and answer data to determine patient output data including one or more possible causes and a recommended level of care, wherein the notation further includes the patient output data.
Example 4: The computer-implemented method of example 3, further comprising: displaying, by the one or more processors and via a patient application, the patient output data and a plurality of options for level of care, including the recommended level of care; receiving, by the one or more processors and via the patient application, a selection of a level of care from the plurality of options for level of care; and based on the selected level of care, providing, by the one or more processors, automatic scheduling of a healthcare visit, wherein the notation further includes a date and time of the healthcare visit scheduled via the patient application.
Example 5: The computer-implemented method of any of the preceding examples, further comprising: providing, by the one or more processors, the question and answer data as input to a trained machine learning system to obtain, as output of the trained machine learning system, a predicted recommendation associated with a care plan for the patient, wherein the notation further includes the predicted recommendation.
Example 6: The computer-implemented method of any of the preceding examples, wherein the referencing comprises: determining a respective complaint identifier for each of the one or more complaints; comparing each respective complaint identifier against the plurality of parent symptoms in the mapping; and verifying each identified parent symptom corresponds to each of the one or more complaints.
Example 7: The computer-implemented method of any of the preceding examples, wherein the categorizing comprises: categorizing a child symptom from the one or more child symptoms in the positive category when a presence of evidence of the child symptom in the question and answer data is determined, wherein the positive category indicates the patient is experiencing the child symptom; and categorizing the child symptom in the negative category when an absence of evidence of the child symptom in the question and answer data is determined, wherein the negative category indicates the patient is not experiencing the child symptom.
Example 8: The computer-implemented method of any of the preceding examples, wherein: each identified parent symptom includes a symptom indicated by the patient during the automated interview, and each of the one or more child symptoms associated with each identified parent symptom corresponds to an answer selectable by the patient in response to one or more questions about the indicated symptom.
Example 9: The computer-implemented method of any of the preceding examples, wherein the graphical format is a tabular format comprised of one or more rows representing the one or more complaints, a first column representing the positive list, and a second column representing the negative list.
Example 10: The computer-implemented method of any of the preceding examples, wherein: the one or more complaints include two complaints, and the extracting, referencing, determining, categorizing, and generating are performed in parallel for each of the two complaints.
Example 11. A system comprising: one or more processors; and at least one memory storing instructions that, when executed by the one or more processors, cause the one or more processors to perform operations including: receiving question and answer data from an automated interview of a patient; extracting one or more complaints from the question and answer data; referencing a mapping that includes a plurality of parent symptoms and a plurality of child symptoms associated with the plurality of parent symptoms to identify a respective parent symptom from the plurality of parent symptoms corresponding to each of the one or more complaints; determining, based on the question and answer data, a presence or absence of evidence of one or more child symptoms, among the plurality of child symptoms, associated with each identified parent symptom; categorizing each of the one or more child symptoms into a positive category or a negative category based on the determination; generating an object that includes, for each of the one or more complaints, (a) a positive list including any of the one or more child symptoms included in the positive category and (b) a negative list including any of the one or more child symptoms included in the negative category; processing the object to generate a history of present illness (HPI) portion of a notation for the patient in a graphical format; storing the notation in an electronic health record of the patient; and generating and transmitting, to a healthcare provider application, a notification that includes one or more indications of an occurrence of the automated interview and availability of the notation in the electronic health record for access.
Example 12: The system of example 11, the operations further comprising: processing the question and answer data using one or more natural language generation (NLG) processing techniques to generate a summary format of the question and answer data, wherein the notation further includes the summary format.
Example 13: The system of example 10 or 11, the operations further comprising: processing the question and answer data to determine patient output data including one or more possible causes and a recommended level of care, wherein the notation further includes the patient output data.
Example 14. The system of example 13, the operations further comprising: displaying, by the one or more processors and via a patient application, the patient output data and a plurality of options for level of care, including the recommended level of care; receiving a selection of a level of care from the plurality of options for level of care provided; and based on the selected level of care, providing automatic scheduling of a healthcare visit, wherein the notation further includes a date and time of the healthcare visit scheduled via the patient application.
Example 15. The system of any of the preceding examples, the operations further comprising: providing the question and answer data as input to a trained machine learning system to obtain, as output of the trained machine learning system, a predicted recommendation associated with a care plan for the patient, wherein the notation further includes the predicted recommendation.
Example 16. The system of any of the preceding examples, wherein the categorizing comprises: categorizing a child symptom from the one or more child symptoms in the positive category when a presence of evidence of the child symptom in the question and answer data is determined, wherein the positive category indicates the patient is experiencing the child symptom; and categorizing the child symptom in the negative category when an absence of evidence of the child symptom in the question and answer data is determined, wherein the negative category indicates the patient is not experiencing the child symptom.
Example 17. The system of any of the preceding examples, wherein: each identified parent symptom includes a symptom indicated by the patient during the automated interview, and each of the one or more child symptoms associated with each identified parent symptom corresponds to an answer selectable by the patient in response to one or more questions about the indicated symptom.
Example 18. The system of any of the preceding examples, wherein the graphical format is a tabular format comprised of one or more rows representing the one or more complaints, a first column representing the positive list, and a second column representing the negative list.
Example 19. The system of any of the preceding examples, wherein: the one or more processors are configured to perform multi-threading, the one or complaints include two complaints, and the referencing, determining, categorizing, and generating are performed in parallel for each of the two complaints via the multi-threading.
Example 20. A non-transitory computer readable medium storing instructions which, when executed by one or more processors, cause the one or more processors to perform operations comprising: receiving question and answer data from an automated interview of a patient; extracting one or more complaints from the question and answer data; referencing a mapping that includes a plurality of parent symptoms and a plurality of child symptoms associated with the plurality of parent symptoms to identify a respective parent symptom from the plurality of parent symptoms corresponding to each of the one or more complaints; determining, based on the question and answer data, a presence or absence of evidence of one or more child symptoms, among the plurality of child symptoms, associated with each identified parent symptom; categorizing each of the one or more child symptoms into a positive category or a negative category based on the determination; generating an object that includes, for each of the one or more complaints, (a) a positive list including any of the one or more child symptoms included in the positive category and (b) a negative list including any of the one or more child symptoms included in the negative category; processing the object to generate a history of present illness (HPI) portion of a notation for the patient in a graphical format; storing the notation in an electronic health record of the patient; and generating and transmitting, to a healthcare provider application, a notification that includes one or more indications of an occurrence of the automated interview and availability of the notation in the electronic health record for access.

What is claimed is:

1. A computer-implemented method for automatic health data processing, the method comprising:
receiving, by one or more processors, question and answer data from an automated interview of a patient;
extracting, by the one or more processors, one or more complaints from the question and answer data;
referencing, by the one or more processors, a mapping that includes a plurality of parent symptoms and a plurality of child symptoms associated with the plurality of parent symptoms, to identify a respective parent symptom from the plurality of parent symptoms corresponding to each of the one or more complaints;
determining, by the one or more processors and based on the question and answer data, a presence or absence of evidence of one or more child symptoms, among the plurality of child symptoms, associated with each identified parent symptom;
categorizing, by the one or more processors, each of the one or more child symptoms into a positive category or a negative category based on the determination;
generating, by the one or more processors, an object that includes, for each of the one or more complaints, (a) a positive list including any of the one or more child symptoms included in the positive category and (b) a negative list including any of the one or more child symptoms included in the negative category;
processing, by the one or more processors, the object to generate a history of present illness (HPI) portion of a notation for the patient in a graphical format;
storing, by the one or more processors, the notation in an electronic health record of the patient; and
generating and transmitting, by the one or more processors and to a healthcare provider application, a notification that includes one or more indications of an occurrence of the automated interview and availability of the notation in the electronic health record for access.

2. The computer-implemented method of claim 1, further comprising:
processing, by the one or more processors, the question and answer data using one or more natural language generation (NLG) processing techniques to generate a summary format of the question and answer data, wherein the notation further includes the summary format.

3. The computer-implemented method of claim 1, further comprising:
processing, by the one or more processors, the question and answer data to determine patient output data including one or more possible causes and a recommended level of care, wherein the notation further includes the patient output data.

4. The computer-implemented method of claim 3, further comprising:
displaying, by the one or more processors and via a patient application, the patient output data and a plurality of options for level of care, including the recommended level of care;
receiving, by the one or more processors and via the patient application, a selection of a level of care from the plurality of options for level of care; and
based on the selected level of care, providing, by the one or more processors, automatic scheduling of a healthcare visit, wherein the notation further includes a date and time of the healthcare visit scheduled via the patient application.

5. The computer-implemented method of claim 1, further comprising:
providing, by the one or more processors, the question and answer data as input to a trained machine learning system to obtain, as output of the trained machine learning system, a predicted recommendation associated with a care plan for the patient, wherein the notation further includes the predicted recommendation.

6. The computer-implemented method of claim 1, wherein the referencing comprises:
determining a respective complaint identifier for each of the one or more complaints;
comparing each respective complaint identifier against the plurality of parent symptoms in the mapping; and
verifying each identified parent symptom corresponds to each of the one or more complaints.

7. The computer-implemented method of claim 1, wherein the categorizing comprises:
categorizing a child symptom from the one or more child symptoms in the positive category when a presence of evidence of the child symptom in the question and answer data is determined, wherein the positive category indicates the patient is experiencing the child symptom; and
categorizing the child symptom in the negative category when an absence of evidence of the child symptom in the question and answer data is determined, wherein the negative category indicates the patient is not experiencing the child symptom.

8. The computer-implemented method of claim 1, wherein:
each identified parent symptom includes a symptom indicated by the patient during the automated interview, and
each of the one or more child symptoms associated with each identified parent symptom corresponds to an answer selectable by the patient in response to one or more questions about the indicated symptom.

9. The computer-implemented method of claim 1, wherein the graphical format is a tabular format comprised of one or more rows representing the one or more complaints, a first column representing the positive list, and a second column representing the negative list.

10. The computer-implemented method of claim 1, wherein:
the one or more complaints include two complaints, and
the extracting, referencing, determining, categorizing, and generating are performed in parallel for each of the two complaints.

11. A system for automatic health data processing, the system comprising:
one or more processors; and
at least one memory storing instructions that, when executed by the one or more processors, cause the one or more processors to perform operations including:
receiving question and answer data from an automated interview of a patient;
extracting one or more complaints from the question and answer data;
referencing a mapping that includes a plurality of parent symptoms and a plurality of child symptoms associated with the plurality of parent symptoms to identify a respective parent symptom from the plurality of parent symptoms corresponding to each of the one or more complaints;
determining, based on the question and answer data, a presence or absence of evidence of one or more child symptoms, among the plurality of child symptoms, associated with each identified parent symptom;
categorizing each of the one or more child symptoms into a positive category or a negative category based on the determination;
generating an object that includes, for each of the one or more complaints, (a) a positive list including any of the one or more child symptoms included in the positive category and (b) a negative list including any of the one or more child symptoms included in the negative category;
processing the object to generate a history of present illness (HPI) portion of a notation for the patient in a graphical format;
storing the notation in an electronic health record of the patient; and
generating and transmitting, to a healthcare provider application, a notification that includes one or more indications of an occurrence of the automated interview and availability of the notation in the electronic health record for access.

12. The system of claim 11, the operations further comprising:
processing the question and answer data using one or more natural language generation (NLG) processing techniques to generate a summary format of the question and answer data, wherein the notation further includes the summary format.

13. The system of claim 11, the operations further comprising:
processing the question and answer data to determine patient output data including one or more possible causes and a recommended level of care, wherein the notation further includes the patient output data.

14. The system of claim 13, the operations further comprising:
displaying, by the one or more processors and via a patient application, the patient output data and a plurality of options for level of care, including the recommended level of care;
receiving a selection of a level of care from the plurality of options for level of care provided; and
based on the selected level of care, providing automatic scheduling of a healthcare visit, wherein the notation further includes a date and time of the healthcare visit scheduled via the patient application.

15. The system of claim 11, the operations further comprising:
providing the question and answer data as input to a trained machine learning system to obtain, as output of the trained machine learning system, a predicted recommendation associated with a care plan for the patient, wherein the notation further includes the predicted recommendation.

16. The system of claim 11, wherein the categorizing comprises:
categorizing a child symptom from the one or more child symptoms in the positive category when a presence of evidence of the child symptom in the question and answer data is determined, wherein the positive category indicates the patient is experiencing the child symptom; and
categorizing the child symptom in the negative category when an absence of evidence of the child symptom in the question and answer data is determined, wherein the negative category indicates the patient is not experiencing the child symptom.

17. The system of claim 11, wherein:
each identified parent symptom includes a symptom indicated by the patient during the automated interview, and
each of the one or more child symptoms associated with each identified parent symptom corresponds to an answer selectable by the patient in response to one or more questions about the indicated symptom.

18. The system of claim 11, wherein the graphical format is a tabular format comprised of one or more rows representing the one or more complaints, a first column representing the positive list, and a second column representing the negative list.

19. The system of claim 11, wherein:
the one or more processors are configured to perform multi-threading,
the one or complaints include two complaints, and
the referencing, determining, categorizing, and generating are performed in parallel for each of the two complaints via the multi-threading.

20. A non-transitory computer readable medium for automatic health data processing, the non-transitory computer readable medium storing instructions which, when executed by one or more processors, cause the one or more processors to perform operations comprising:
receiving question and answer data from an automated interview of a patient;
extracting one or more complaints from the question and answer data;
referencing a mapping that includes a plurality of parent symptoms and a plurality of child symptoms associated with the plurality of parent symptoms to identify a respective parent symptom from the plurality of parent symptoms corresponding to each of the one or more complaints;
determining, based on the question and answer data, a presence or absence of evidence of one or more child symptoms, among the plurality of child symptoms, associated with each identified parent symptom;
categorizing each of the one or more child symptoms into a positive category or a negative category based on the determination;
generating an object that includes, for each of the one or more complaints, (a) a positive list including any of the one or more child symptoms included in the positive category and (b) a negative list including any of the one or more child symptoms included in the negative category;
processing the object to generate a history of present illness (HPI) portion of a notation for the patient in a graphical format;
storing the notation in an electronic health record of the patient; and
generating and transmitting, to a healthcare provider application, a notification that includes one or more indications of an occurrence of the automated interview and availability of the notation in the electronic health record for access.
